(19) United States
(12) Patent Application Publication
(10) Pub. No.: US 2014/0202452 A1
(43) Pub. Date: Jul. 24, 2014

(54) SYSTEMS AND METHODS FOR SUGAR REFINING

(71) Applicants: Robert Jansen, Collinsville, IL (US); Aharon Eyal, Jerusalem (IL)

(72) Inventors: Robert Jansen, Collinsville, IL (US); Aharon Eyal, Jerusalem (IL)

(21) Appl. No.: 13/983,761

(22) PCT Filed: Feb. 6, 2012
(86) PCT No.: PCT/US12/24033

§ 371 Date: Feb. 19, 2014

Related U.S. Application Data
(60) Provisional application No. 61/524,839, filed on Aug. 18, 2011.
(60) Provisional application No. 61/533,088, filed on Sep. 9, 2011.
(60) Provisional application No. 61/539,873, filed on Sep. 27, 2011.

(30) Foreign Application Priority Data
Feb. 6, 2011 (IL) 211093

(51) Int. Cl. C13K 1/04

(52) U.S. Cl. CPC C13K 1/04
USPC 127/36

(57) ABSTRACT

A method comprising: (a) extracting a sugar mixture in an aqueous solution of at least 30% HCL/[HCl+water] by weight with an extractant including an S1 solvent; (b) increasing a monomeric sugar to oligomeric sugar ratio in the mixture to produce a monomeric sugar enriched mixture comprising at least 65% monomeric sugars by weight relative to total sugars; and (c) separating an S1/HCl liquid phase comprising more than 30% HCl/[HCl+water] from said sugar mixture.

### RELATED APPLICATIONS

In accord with the provisions of 35 U.S.C. §119(a) and/or §365(b), this application claims priority from:

IL 211093 entitled “A METHOD FOR PROCESSING A LIGNOCELLULOSIC MATERIAL AND FOR THE PRODUCTION OF A CARBOHYDRATE COMPOSITION” to Robert JANSEN et al. filed on Feb. 6, 2011; which is fully incorporated herein by reference.

In accord with the provisions of 35 U.S.C. §119(e) and/or §363, this application claims the benefit of:

U.S. 61/524,839 entitled “SYSTEMS AND METHODS FOR SUGAR REFINING” to Robert JANSEN et al. filed on Aug. 18, 2011;

U.S. 61/533,088 entitled “SYSTEMS AND METHODS FOR SUGAR REFINING” to Robert JANSEN et al. filed on Sep. 9, 2011; and

U.S. 61/539,873 entitled “A METHOD FOR PROCESSING A LIGNOCELLULOSIC MATERIAL” to Robert JANSEN et al. filed on Sep. 27, 2011; each of which is fully incorporated herein by reference.

In addition, this application is related to the following co-pending applications, each of which is fully incorporated herein by reference:

PCT/US2011/064237 entitled “METHODS AND SYSTEMS FOR PROCESSING LIGNOCELLULOSIC MATERIALS AND RELATED COMPOSITIONS” to Aharon EYAL et al. filed on Sep. 12, 2011;

PCT/US2011/057552 entitled “HYDROLYSIS SYSTEMS AND METHODS” to Aharon EYAL et al. filed on Oct. 24, 2011;

PCT/IL2011/000424 entitled “LIGNIN COMPOSITIONS, SYSTEMS AND METHODS FOR PROCESSING LIGNIN AND/OR HCl” to Robert JANSEN et al. filed on Jun. 1, 2011;

PCT/IL2011/000509 entitled “SUGAR MIXTURES AND METHODS FOR PRODUCTION AND USE THEREOF” to Aharon EYAL et al. filed on Jun. 26, 2011;

PCT/IL2011/000517 entitled “METHODS OF PROCESSING A SUCROSE CROP” to Aharon EYAL et al. filed on Jun. 28, 2011; and

PCT/US11/46153 entitled “METHODS AND SYSTEMS FOR SOLVENT PURIFICATION” to Robert JANSEN et al. filed on Aug. 1, 2011.

### FIELD OF THE INVENTION

This invention relates to sugar refining methods and to systems and/or apparatus suitable for use in sugar refining methods.

### BACKGROUND OF THE INVENTION

The carbohydrate-conversion industry currently ferments about 100 million tons of carbohydrates annually to provide fuel-grade ethanol.

Millions of tons of carbohydrates are also fermented every year to provide food and feed products, such as citric acid and lysine.

The carbohydrate-conversion industry also includes fermentation to industrial products, such as monomers for the polymer industry, e.g. lactic acid for the production of polylactide.

Carbohydrates are an attractive and environment-friendly substrate since they are obtained from renewable crop resources. For example sucrose can be produced from sugar canes and glucose can be produced from corn and wheat starches.

However, crop resources such as sugar cane, corn and wheat are produced primarily for human consumption and/or as livestock feed. Increased consumption of these crop resources by the carbohydrate-conversion industry may impact food costs.

As an alternative, many renewable non-food resources are potential sources of soluble carbohydrates. The renewable non-food resources can generally be described as “woody materials” or “lignocellulosic materials”. Woody materials include wood and by-products of wood processing (e.g. sawdust, shavings) as well as residual plant material from agricultural products.

Residual plant material from agricultural products includes processing by-products and field remains.

Processing by-products include, but are not limited to, corn cobs, sugar cane bagasse, sugar beet pulp, empty fruit bunches from palm oil production, straw (e.g. wheat or rice), soy bean hulls, residual meals from the vegetable oil industry (e.g. soybean, peanut, corn or rapeseed) wheat bran and fermentation residue from the beer and wine industries.

Field remains includes, but, is not limited to, corn stover, post-harvest cotton plants, post-harvest soybean bushes and post-harvest rapeseed plants.

Woody materials also include “energy crops” such as switch grass and/or broom grass, which grow rapidly and generate low-cost biomass specifically as a source of carbohydrates.

These woody materials contain cellulose, hemicellulose and lignin as their main components and are also referred to as lignocellulosic material. These lignocellulosic materials also contain mineral salts (ashes) and organic compounds, such as tall oils. The degree and type of these non-carbohydrate materials can create technical problems in production of soluble carbohydrates.

Despite the theoretical feasibility of realizing useful sugars from these renewable non-food resources, actual industrial production of such sugars has been limited.

This application refers to various solvents defined in terms of Hoy's cohesion parameter Delta-P and/or Delta-H. By way of review:

Delta-P is the polarity related component of Hoy's cohesion parameter and delta-H is the hydrogen bonding related component of Hoy's cohesion parameter.

The cohesion parameter, as referred to above or, solubility parameter, was defined by Hildebrand as the square root of the cohesive energy density:

$$δ = \sqrt{\frac{Δ {E}_{vap}}{V}}$$

where ΔEvap and V are the energy or heat of vaporization and molar volume of the liquid, respectively. Hansen extended the original Hildebrand parameter to a three-dimensional cohesion parameter. According to this concept, the total solubility parameter, delta, is separated into three different components, or, partial solubility parameters relating to the specific intermolecular interactions:

δ²−δ_{d}²+δₚ²+δₕ²

in which delta-D, delta-P and delta-H are the dispersion, polarity, and hydrogen bonding components, respectively. Hoy proposed a system to estimate total and partial solubility parameters. The unit used for those parameters is MPa^{1/2}. A detailed explanation of that parameter and its components can be found in “CRC Handbook of Solubility Parameters and Other Cohesion Parameters”, second edition, pages 122-138. That and other references provide tables with the parameters for many compounds. In addition, methods for calculating those parameters are provided.

### SUMMARY OF THE INVENTION

A broad aspect of the invention relates to sugar refining. As used in this specification and the accompanying claims, the term “sugar refining” relates to one or more chemical engineering processes and/or operations which transform an input sugar composition with a relatively low economic value to a refined output sugar composition with a relatively high economic value. According to various exemplary embodiments of the invention the output sugar composition can be provided as a concentrated solution and/or syrup and/or crude crystals and/or purified crystals.

One aspect of some embodiments of the invention relates to use of an organic solvent to separate HCl from an input sugar composition provided in a solution of concentrated HCl.

In some exemplary embodiments of the invention, the organic solvent is an “S1” solvent. As used in this specification and the accompanying claims, an “S1” solvent (S1) is a solvent characterized by a water solubility of less than 15% wt, optionally less than 10% wt, optionally less than 5% wt and optionally less than 1% wt and by a delta-P between 5 and 10 MPa^{1/2} and/or a delta-H between 5 and 20 MPa^{1/2}. According to various exemplary embodiments of the invention a specific S1 solvent is selected based upon its ability to selectively extract HCl from an aqueous sugar solution.

Alternatively or additionally, the solubility of water in S1 is less than 20% wt, optionally less than 15% wt, optionally less than 10% wt and optionally less than 8% wt. As used in this specification and the accompanying claims the “solubility” is measured by the percent weight ratio (wt %) and determined by combining an essentially pure solvent and de-ionized water at 25° C., and measuring the wt % of the solvent dissolved in the water, or of the water dissolved in the solvent.

In some exemplary embodiments of the invention, S1 has a boiling point at 1 atm between 100° C. and 200° C. and forms a heterogeneous azeotrope with water having a boiling point at 1 atm of less than 100° C. Optionally, these physical properties contribute to an ease of recovery and/or recycling of HCl. Optionally, recovery processes include distillation.

In some exemplary embodiments of the invention, S1 includes one or more alcohols and/or ketones and/or aldehydes having at least 5 carbon atoms.

Optionally, S1 includes one or more of pentanols, hexanols, heptanols, octanols, nonanols, decanols, methyl-isobutyl-ketone, methyl-butyl-ketone and combinations thereof. As used herein, the term alcohols means any of mono-, di- and poly-alcohols, primary, secondary and tertiary alcohols, straight chain and branched chain alcohols and any combination thereof. According to particular exemplary embodiments, S1 is selected from hexanol and 2-ethyl-1-hexanol. In some exemplary embodiments of the invention, S1 includes only n-hexanol. In other exemplary embodiments of the invention, S1 includes only 2-ethyl-hexanol. Optionally, n-hexanol or 2-ethyl-1-hexanol is combined with another non-hexanol solvent.

According to various exemplary embodiments of the invention the input sugar composition is in excess of 30%, optionally in excess of 33%, optionally in excess of 35%, optionally in excess of 37% HCl/[HCl+water] by weight. In some exemplary embodiments of the invention, the extractant including organic solvent is applied to the input sugar composition in a countercurrent stream. In some exemplary embodiments of the invention, the extractant including the organic solvent is applied in two or more separate extraction operations. Optionally, a subsequent extraction employs extractant reserved from a previous extraction.

The terms “extracting” and “extraction” and grammatical variations thereof as used in this specification and the accompanying claims indicate contacting between a liquid extractant and another liquid containing material. The result of such an extraction is transfer of one or more materials to the liquid extractant in a selective manner. By way of example, S1 is employed in some exemplary embodiments of the invention to extract HCl and/or water from a sugar composition. According to such exemplary embodiments, HCl is extracted from sugars, optionally with some water. According to various exemplary embodiments of the invention this initial extraction partially de-acidifies the sugar mixture and further processing is performed to remove residual HCl from the sugars. Optionally, this further processing includes chromatographic separation.

The terms “soluble in” and “solubility” and grammatical variations thereof as used in this specification and the accompanying claims indicate solubility of a first substance in a second substance at 25° C.

Throughout this specification and accompanying claims the terms “soluble carbohydrate” and “soluble sugar” each indicate solubility in water and/or the described aqueous HCl solutions at 25° C. Soluble carbohydrates or soluble sugars are present in a sugar mixture including monomeric sugars and oligomeric sugars of relatively short chain length (dimers and higher oligosaccharides including from 3 to about 10 or 11 saccharide units).

According to various exemplary embodiments of the invention at least about 80%, optionally at least about 85%, optionally at least about 90%, optionally at least about 95% and optionally about 100% of the oligomeric sugars in the mixture are water soluble.

For many downstream applications, an increase in the percentage of monomeric sugars in the mixture contributes to an increase in economic value. In some cases the increase in economic value is reflective of an increase in process efficiency. For example, many micro-organisms can only convert monomeric sugars to ethanol.

In some exemplary embodiments of the invention, a hydrolyzate is described as being extracted. According to various exemplary embodiments of the invention this extraction may be on the hydrolyzate per se or on a modified hydrolyzate. Optional modifications include, but are not limited to, dilution, concentration, mixing with another stream, temperature adjustment, a chemical conversion of a component (e.g. hydrolysis of oligomers), removing of components (e.g. via ion-exchange resin) and filtration. Optionally, two or more modifications may be performed prior to extraction.

In some exemplary embodiments of the invention, HCl selectively transfers to the extractant during extraction to form an HCl-carrying extract and an HCl-depleted stream. Optionally, HCl is recovered from the extract and/or the HCl-depleted stream. In some exemplary embodiments of the invention, recovered HCl is recycled.

Another aspect of some embodiments of the invention relates to reduction of a concentration of oligomeric sugars (i.e. dimers and/or higher oligosaccharides) out of total sugars in the sugar composition.

In some exemplary embodiments of the invention, hydrolysis in a dilute acid, such as dilute HCl contributes to this reduction. Optionally, the dilute HCl is provided at a concentration of 5 to 15%, optionally 7 to 13%, optionally 9 to 11%, optionally about 10%. Alternatively or additionally, chromatography (e.g. using ion exchange resin) contributes to this reduction. In some exemplary embodiments of the invention, hydrolysis conditions are adjusted to reduce a tendency of monomeric sugars to re-oligomerize. Optionally, the time during which hydrolysis continues, and/or the temperature is adjusted and these factors contribute to the reduced tendency of monomeric sugars to re-oligomerize.

Another aspect of some embodiments of the invention relates to separation of monomeric sugars in the sugar composition from HCl and/or oligomeric sugars. In some exemplary embodiments of the invention, chromatography, optionally using an ion exchange resin, contributes to this separation. In some exemplary embodiments of the invention, separated HCl and oligomeric sugars are recycled to the hydrolysis in dilute acid mentioned above. According to various exemplary embodiments of the invention chromatography resins including, but not limited to, strong and/or weak cation exchangers in acid or salt form and/or strong and/or weak anion exchangers in free base and/or salt form can be employed for this separation.

The term “feeding” and grammatical variations thereof as used in the context of chromatography throughout this specification and the accompanying claims indicates application of a sample so that the sample contacts the resin.

Yet another aspect of some embodiments of the invention relates to a sugar refinery designed and configured to process an input sugar composition provided as 20 to 35% (optionally about 30%) sugar in concentrated HCl (e.g. 30% HCl/[HCl+water] by weight). In some exemplary embodiments of the invention, the input sugar composition includes as much as 50, 60 or 70% or intermediate or greater percentages of oligomeric sugars and as little as 50, 40 or 30% or intermediate or lesser amounts of monomeric sugars. In some exemplary embodiments of the invention, the refined output sugar composition includes more than 80%, optionally more than 90%, optionally more than 92%, optionally more than 93%, optionally 95% or more monomeric sugars (relative to total sugars) Optionally, the refined output sugar is provided as a “syrup” with a sugar concentration of at least 40%, optionally at least 50%, optionally at least 60%, optionally at least 70%, optionally 80% or more in water. In other exemplary embodiments of the invention, the refined output sugar is provided as crystals.

According to various exemplary embodiments of the invention, two or more aspects are combined to provide a synergistic effect. Optionally, recycling of materials (e.g. effluents and/or washes) from one process to another contributes to this synergy.

It will be appreciated that the various aspects described above relate to solution of technical problems which are unique to input sugar compositions received from acid hydrolysis of lignocellulosic substrates (i.e. woody materials).

Alternatively or additionally, it will be appreciated that the various aspects described above relate to solution of technical problems related to downstream biological processes which are ill suited to handle dimeric sugars or longer oligomers of sugars.

Although conversion of lignocellulosic material to carbohydrates via enzyme-catalyzed and/or acid-catalyzed hydrolysis of polysaccharides has been previously described, attempts at industrial scale application of the proposed technologies have been largely unsuccessful.

This technical problem is relevant to downstream applications including, but not limited to fermentation and chemical conversion. For example, micro-organisms used in fermentation typically exhibit a strong preference for specific sugar monomers and/or are unable to break down trisaccharides or longer oligosaccharides. Alternatively or additionally, some chemical conversions of industrially useful monomers may not be able to convert dimers or higher polymers in the feedstock. This means that the presence of non-monomeric sugars can potentially have a negative impact on downstream production of bio-fuels (e.g. ethanol), use in the food industry (e.g. conversion of xylose to xylitol for use as an artificial sweetener) and lactic acid to polylactide.

In some exemplary embodiments of the invention, there is provided a method including: (a) extracting a sugar mixture in an aqueous solution of at least 30% HC/[HCl+water] by weight with an extractant including an S1 solvent; (b) increasing a monomeric sugar to oligomeric sugar ratio in the mixture to produce a monomeric sugar enriched mixture including at least 65% monomeric sugars by weight relative to total sugars; and (c) separating an S1/HCl liquid phase including more than 30% HCl/[HCl+water] from the sugar mixture. In some embodiments, the S1 solvent includes a single member of the group consisting of n-hexanol and 2-ethyl-hexanol. Alternatively or additionally, in some embodiments, the S1 solvent consists essentially of n-hexanol. Alternatively or additionally, in some embodiments the S1 solvent consists essentially of 2-ethyl-hexanol. Alternatively or additionally, in some embodiments the increasing includes performing chromatographic separation. Alternatively or additionally, in some embodiments the extracting includes at least two extraction operations. Alternatively or additionally, in some embodiments the monomeric-sugar enriched mixture includes ≧30% total sugar. Alternatively or additionally, in some embodiments the method includes hydrolyzing oligomeric sugars to monomeric sugars between a pair of the at least two extraction operations. Alternatively or additionally, in some embodiments at least one of the at least two extraction operations employs an HCl-containing extract from a previous extraction operation as an extractant. Alternatively or additionally, in some embodiments the chromatographic separation produces an acid cut enriched in oligomeric sugars relative to the sugar mixture and a monomer cut enriched in monomeric sugars relative to the sugar mixture on a weight basis. Alternatively or additionally, in some embodiments the method includes separating HCl from S1 by distillation.

In some exemplary embodiments of the invention, there is provided a method including: (a) feeding a resin in a chromatographic mode with a sugar mixture including monomeric and oligomeric sugars in 4 to 8% HCl; and (b) feeding the resin with an aqueous solution to produce an acid cut enriched in oligomeric sugars relative to total sugars and a monomer cut enriched in monomeric sugars relative to total sugars compared to the sugar mixture on a weight basis. In some embodiments, the mixture includes 45 to 63% total sugars by weight. Alternatively or additionally, in some embodiments the method includes hydrolyzing oligomeric sugars in the acid cut to produce a secondary hydrolyzate enriched with monomeric sugars (relative to total sugars). Alternatively or additionally, in some embodiments the method includes incorporation of sugars from the secondary hydrolyzate into the sugar mixture. Alternatively or additionally, in some embodiments the hydrolyzing is catalyzed by HCl at a concentration of not more than 10%. Alternatively or additionally, in some embodiments the hydrolyzing is performed at a temperature not exceeding 97° C. Alternatively or additionally, in some embodiments the secondary hydrolyzate contains at least 65; 70; 75; 80 or even 85% or intermediate or greater percentages by weight monomeric sugars out of total sugars. Alternatively or additionally, in some embodiments the total sugar content of the secondary hydrolyzate is at least 95% by weight of the sugar content of the mixture. Alternatively or additionally, in some embodiments the monomer cut contains at least 80% by weight monomeric sugars out of total sugars.

In some exemplary embodiments of the invention, there is provided a method including: (a) providing a mixture of oligomeric and monomeric sugars at a total concentration of at least 30% in an aqueous solution of at least 10% HCl; (b) reducing the sugar concentration below 25%; and (c) hydrolyzing oligomeric sugars in the mixture to produce a hydrolyzate enriched with monomeric sugars (relative to total sugars). In some embodiments, the hydrolyzing is catalyzed by HCl at a concentration of not more than 10% HCl by weight. Alternatively or additionally, in some embodiments the hydrolyzing is performed at a temperature not exceeding 97° C. Alternatively or additionally, in some embodiments the secondary hydrolyzate contains at least 65; 70; 75; 80 or even 85% or intermediate or greater percentages monomeric sugars out of total sugars. Alternatively or additionally, in some embodiments the total sugar content of the secondary hydrolyzate is at least 95% by weight of the sugar content of the mixture. Alternatively or additionally, in some embodiments the method includes evaporating water from the hydrolyzate at a temperature not exceeding 70° C. Alternatively or additionally, in some embodiments less than 10% of monomeric sugars in the hydrolyzate oligomerize during the evaporation. Alternatively or additionally, in some embodiments the method includes including extracting the secondary hydrolyzate with an extractant including S1 solvent to produce an extracted hydrolyzate including not more than 7% HCl by weight. Alternatively or additionally, in some embodiments the extracted hydrolyzate includes at least 50% total sugars by weight. Alternatively or additionally, in some embodiments the method includes feeding a resin in a chromatographic mode with the extracted hydrolyzate, and feeding the resin with an aqueous solution to produce an acid cut enriched in oligomeric sugars relative to total sugars and a monomer cut enriched in monomeric sugars relative to total sugars as compared to the hydrolyzate. Alternatively or additionally, in some embodiments the method is performed cyclically so that the mixture of oligomeric and monomeric sugars includes sugars from a previous acid cut.

In some exemplary embodiments of the invention, there is provided a method including: (a) extracting a sugar mixture in an aqueous solution of at least 30% HCl/[HCl+water] by weight with an extractant including an S1 solvent, wherein extraction involves at least two extraction operations; (b) increasing a monomeric sugar to oligomeric sugar ratio in the mixture to produce a monomeric-sugar enriched mixture including at least 65% monomeric sugars by weight relative to total sugars, and (c) separating an extract including more than 25% HCl/[HCl+water] and a sugar mixture including at least 70% monomeric sugars relative to total sugars and having sugars concentration greater than 40%. In some embodiments, the increasing includes chromatographic separation. Alternatively or additionally, the increasing includes hydrolysis of oligomeric sugars. In some embodiments, the increasing includes chromatographic separation and hydrolysis of oligomeric sugars. Alternatively or additionally, in some embodiments the method includes including at least one internal cycle. In some embodiments, the method includes at least two internal cycles.

In some exemplary embodiments of the invention, there is provided a method including: (a) providing a fermentor; and (b) fermenting a medium including a monomeric-sugar enriched mixture according as described above and/or, a monomer cut according as described above and/or a secondary hydrolyzate enriched with monomeric sugars as described above in the fermentor to produce a conversion product. In some exemplary embodiments of the invention, there is provided method including: (a) providing a monomeric sugar enriched mixture as described above and/or a monomer cut according as described above and/or a secondary hydrolyzate enriched with monomeric sugars as described above; and (b) converting sugars in the at least one member to a conversion product using a chemical process. In some embodiments, the conversion product includes at least one member selected from the group consisting of alcohols, carboxylic acids, amino acids, monomers for the polymer industry and proteins. Alternatively or additionally, in some embodiments the method includes processing the conversion product to produce a consumer product selected from the group consisting of detergent, polyethylene-based products, polypropylene-based products, polyolefin-based products, polylactic acid (polylactide)-based products, polyhydroxyalkanoate-based products and polyacrylic-based products. In some embodiments, the detergent includes a sugar-based surfactant, a fatty acid-based surfactant, a fatty alcohol-based surfactant, or a cell-culture derived enzyme. Alternatively or additionally, in some embodiments the polyacrylic-based products are selected the group consisting of plastics, floor polishes, carpets, paints, coatings, adhesives, dispersions, flocculants, elastomers, acrylic glass, absorbent articles, incontinence pads, sanitary napkins, feminine hygiene products and diapers. Alternatively or additionally, in some embodiments the polyolefin-based products are selected from the group consisting of milk jugs, detergent bottles, margarine tubs, garbage containers, water pipes, absorbent articles, diapers, non-wovens, HDPE toys and HDPE detergent packagings. Alternatively or additionally, in some embodiments the polypropylene-based products are selected from the group consisting of absorbent articles, diapers, and non-wovens. Alternatively or additionally, in some embodiments the polylactic acid-based products are selected from the group consisting of packaging of agriculture products and of dairy products, plastic bottles, biodegradable products and disposables. Alternatively or additionally, in some embodiments the polyhydroxyalkanoate-based products are selected from the group consisting of packaging of agriculture products, plastic bottles, coated papers, molded or extruded articles, feminine hygiene products, tampon applicators, absorbent articles, disposable non-wovens, wipes, medical surgical garments, adhesives, elastomers, films, coatings, aqueous dispersants, fibers, intermediates of pharmaceuticals and binders. Alternatively or additionally, in some embodiments the conversion product includes at least one member selected from the group consisting of ethanol, butanol, isobutanol, a fatty acid, a fatty acid ester, a fatty alcohol and biodiesel. Alternatively or additionally, in some embodiments the method includes processing of the conversion product to produce at least one product selected from the group consisting of an isobutene condensation product, jet fuel, gasoline, gasohol, diesel fuel, drop-in fuel, diesel fuel additive and a precursor thereof. In some embodiments, the gasohol is ethanol-enriched gasoline or butanol-enriched gasoline. Alternatively or additionally, in some embodiments the product is selected from the group consisting of diesel fuel, gasoline, jet fuel and drop-in fuels. Some embodiments of the invention relate to a consumer product, a precursor of a consumer product, or an ingredient of a consumer product produced from a conversion product as described above. Some exemplary embodiments of the invention relate to a consumer product, a precursor of a consumer product, or an ingredient of a consumer product including at least one conversion product produced by a method as described above, wherein the conversion product is selected from the group consisting of carboxylic and fatty acids, dicarboxylic acids, hydroxylcarboxylic acids, hydroxyl di-carboxylic acids, hydroxyl-fatty acids, methylglyoxal, mono-, di-, or poly-alcohols, alkanes, alkenes, aromatics, aldehydes, ketones, esters, biopolymers, proteins, peptides, amino acids, vitamins, antibiotics and pharmaceuticals. In some embodiments, the product is ethanol-enriched gasoline, jet fuel, or biodiesel. Some exemplary embodiments of the invention relate to a consumer product, a precursor of a consumer product, or an ingredient of a consumer product as described above, wherein the consumer product has a ratio of carbon-14 to carbon-12 of about 2.0×10⁻¹³ or greater. Alternatively or additionally some exemplary embodiments of the invention relate to a consumer product including an ingredient as described above and an additional ingredient produced from a raw material other than lignocellulosic material. In some embodiments, the ingredient and the additional ingredient produced from a raw material other than lignocellulosic material are essentially of the same chemical composition. Alternatively or additionally, in some embodiments, the consumer product as described above includes a marker molecule at a concentration of at least 100 ppb. In some embodiments, the marker molecule is selected from the group consisting of furfural, hydroxymethylfurfural, products of furfural or hydroxymethylfurfural condensation, color compounds derived from sugar caramelization, levulinic acid, acetic acid, methanol, galacturonic acid and glycerol.

In some exemplary embodiments of the invention, there is provided a system including: (a) an acid extractor adapted to extract acid from an input stream of at least 20% sugar in an aqueous solution of at least 30% HCl/[HCl+water] with an extractant including an S1 solvent to produce an output sugar stream; and (b) a chromatography component adapted to separate residual acid from sugars in the output stream and produce an acid depleted sugar stream. In some embodiments, the chromatography component includes an ion exchange resin. Alternatively or additionally, in some embodiments the acid extractor includes at least one pulsed column. Alternatively or additionally, in some embodiments the system includes an acid return loop adapted to route the residual acid to the acid extractor. Alternatively or additionally, in some embodiments the acid extractor includes at least two acid extractors arranged in series. Alternatively or additionally, in some embodiments the system includes a secondary hydrolysis reactor disposed between any pair of the at least two acid extractors. Alternatively or additionally, in some embodiments the system includes a filtration unit disposed between any pair of the at least two acid extractors. Alternatively or additionally, in some embodiments the system includes an evaporation unit disposed between any pair of the at least two acid extractors. Alternatively or additionally, in some embodiments water produced by the evaporation unit serves as an elution flow for the chromatography component. Alternatively or additionally, in some embodiments the system includes a desolventizer adapted to remove residual solvent from the acid depleted sugar stream. Alternatively or additionally, in some embodiments the system includes a purification media adapted to remove impurities likely to adversely affect downstream fermentation. Alternatively or additionally, in some embodiments the system includes a concentrator adapted to increase a solids content of the acid depleted sugar stream.

Unless otherwise defined, all technical and scientific terms used herein have the same meaning as commonly understood by one of ordinary skill in the art to which this invention belongs. Although suitable methods and materials are described below, methods and materials similar or equivalent to those described herein can be used in the practice of the present invention. In case of conflict, the patent specification, including definitions, will control. All materials, methods, and examples are illustrative only and are not intended to be limiting.

As used herein, the terms “comprising” and “including” or grammatical variants thereof are to be taken as specifying inclusion of the stated features, integers, actions or components without precluding the addition of one or more additional features, integers, actions, components or groups thereof.

The term “method” refers to manners, means, techniques and procedures for accomplishing a given task including, but not limited to, those manners, means, techniques and procedures either known to, or readily developed from known manners, means, techniques and procedures by practitioners of chemistry and/or engineering.

As used in this specification and the accompanying claims the term “adapted” indicates a modification to a previously recited component to achieve the described function.

Percentages (%) and ratios are W/W (weight per weight) unless otherwise indicated.

### BRIEF DESCRIPTION OF THE DRAWINGS

In order to understand the invention and to see how it may be carried out in practice, embodiments will now be described, by way of non-limiting example only, with reference to the accompanying figures. In the figures, identical and similar structures, elements or parts thereof that appear in more than one figure are generally labeled with the same or similar references in the figures in which they appear. Dimensions of components and features shown in the figures are chosen primarily for convenience and clarity of presentation and are not necessarily to scale. In schematic representations, dimension may be distorted and/or changed from drawing to drawing. The attached figures are:

FIG. 1 is a schematic overview of a system according to some exemplary embodiments of the invention;

FIGS. 2a and 2b are schematic overviews of two different de-acidification systems in accord with some exemplary embodiments of the invention:

FIG. 2c is a schematic overview of an optional solvent and/or water removal system according to some exemplary embodiments of the invention;

FIGS. 3, 4 and 5 are simplified flow diagrams of methods according to various exemplary embodiments of the invention;

FIG. 6 is a schematic representation of a system similar to that in FIG. 2b indicating flow control components;

FIGS. 7a and 7b are simplified flow diagrams of methods according to various exemplary embodiments of the invention; and

FIG. 8 is a simplified flow diagram of a method according to some exemplary embodiments of the invention.

### DETAILED DESCRIPTION OF EMBODIMENTS

Embodiments of the invention relate to systems and methods for refining sugar. Specifically, some embodiments of the invention can be used to separate sugar from an input sugar composition including a mineral acid, such as HCl or H₂SO₄. Alternatively or additionally, some embodiments of the invention can be used to produce an output sugar composition which has a higher proportion of monomeric sugars (relative to total sugars) than the input sugar composition.

The principles and operation of a system and/or methods according to exemplary embodiments of the invention may be better understood with reference to the drawings and accompanying descriptions.

Before explaining at least one embodiment of the invention in detail, it is to be understood that the invention is not limited in its application to the details set forth in the following description. The invention is capable of other embodiments or of being practiced or carried out in various ways. Also, it is to be understood that the phraseology and terminology employed herein is solely descriptive, as opposed to limiting.

Overview of Exemplary System

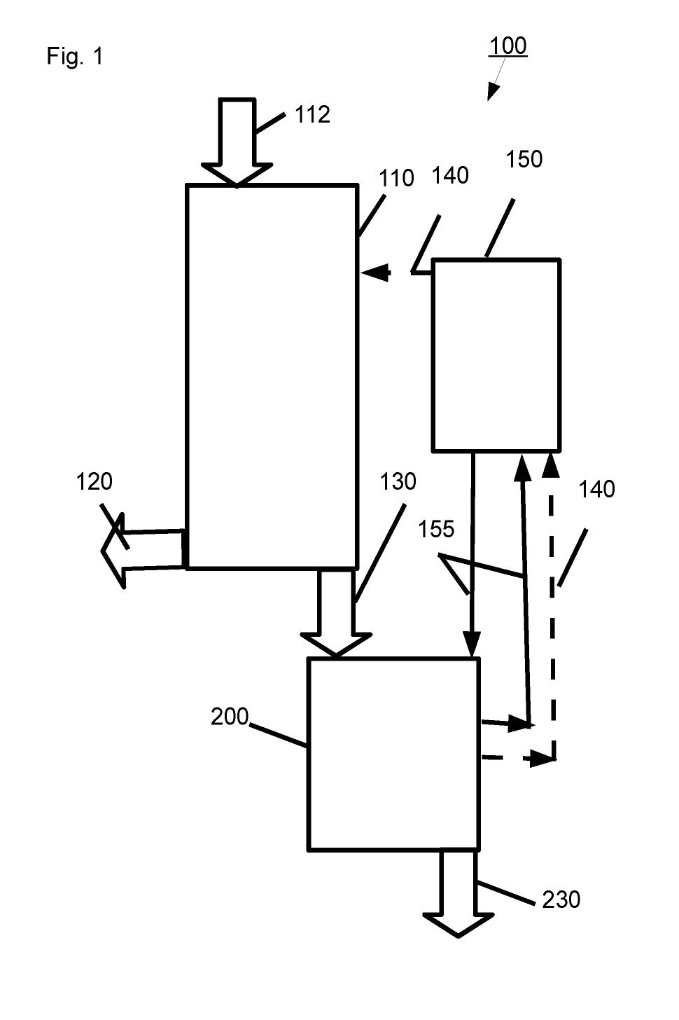

FIG. 1 is a simplified schematic diagram of a system for hydrolysis of a lignocellulosic substrate indicated generally as 160. Depicted system 100 includes a main hydrolysis reactor 110 adapted to receive a lignocellulosic substrate input 112. Optionally, substrate 112 is provided as wood chips, although any “woody material” as described in the background can be used instead of wood. Reactor 110 can be adapted for any type of hydrolysis. For purposes of illustration, a description of an exemplary embodiment which uses concentrated acid (e.g. HCl or H₂SO₄) in reactor 110 is provided. This illustration does not limit the scope of the invention.

In the depicted exemplary system 100, substrate 112 is brought into contact with a concentrated acid (e.g. HCl) solution in reactor 110 and hemicellulose and/or cellulose in the substrate are hydrolyzed to produce a mixture of soluble sugars and residual lignin. These materials are collected separately as lignin stream 120 and sugar mixture 130, each of which contains a large amount of residual acid.

This application is primarily concerned with processing of sugar mixture 130. The processing includes removal of the residual acid as well as modification of the mixture to convert oligomeric sugars to monomeric sugars. This processing is conducted in a sugar refining module, designated here as 200.

As will be explained in greater detail hereinbelow, refining module 200 employs a flow of extractant including organic solvent 155 (solid arrows) to extract HCl 140 (dashed arrows) from sugar mixture 130.

Refined sugars 230 are the primary product of refining module 200. Module 200 also produces a stream of acid (e.g. HCl) 140 mixed with solvent 155 (depicted as parallel dashed and solid arrows respectively for clarity) which is routed to a solvent/HCl recovery module 150. Recovery module 150 separates acid 140 from solvent 155. In some exemplary embodiments of the invention, this separation of acid from solvent is by distillation. In some embodiments, acid 140 is recycled to hydrolysis reactor 110 and/or solvent 155 is recycled to refining module 200.

Refined sugars 230 can be used in a wide variety of subsequent industrial processes. For example, refined sugars 230 can be fermented to produce ethanol, optionally for use as a fuel.

Exemplary Sugar Refining System

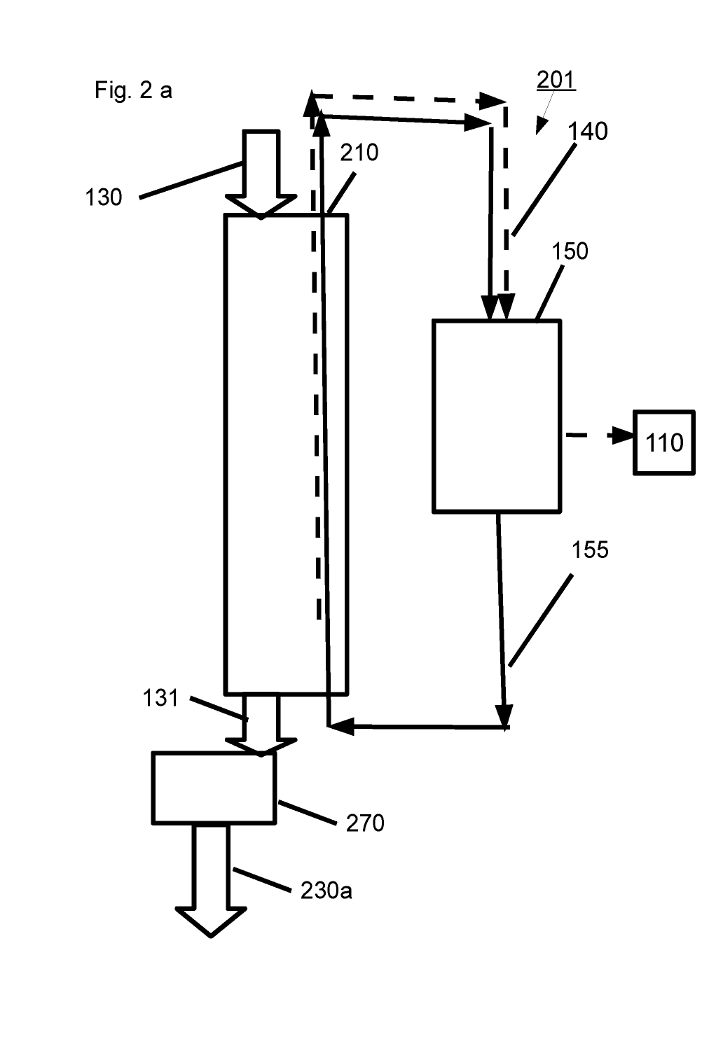

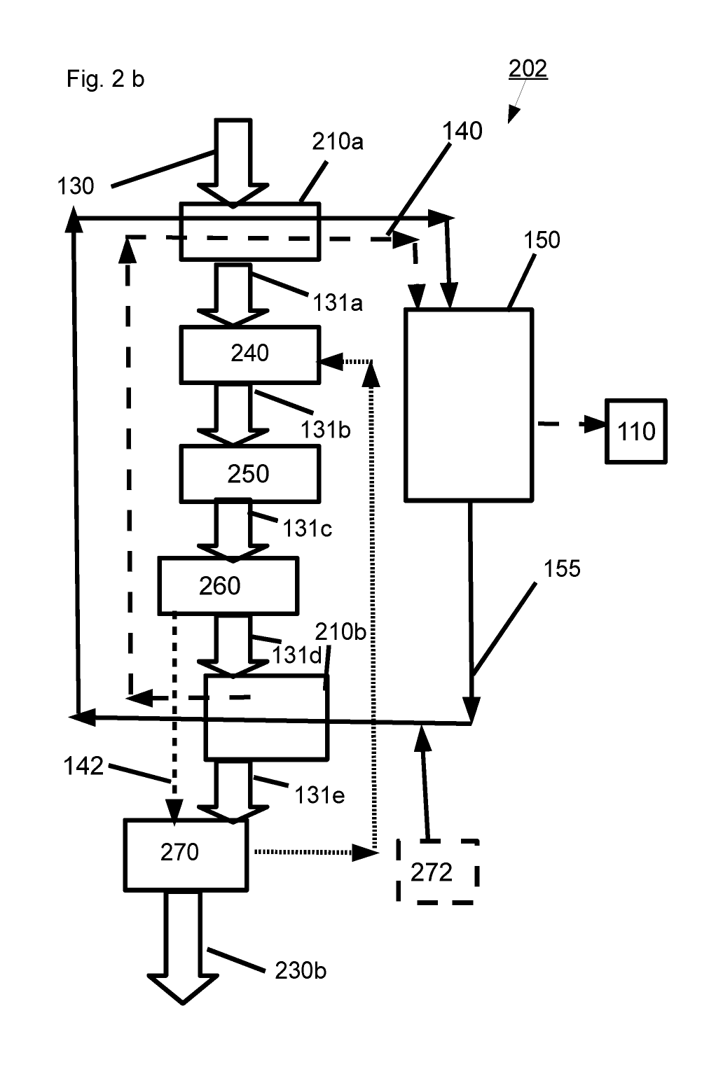

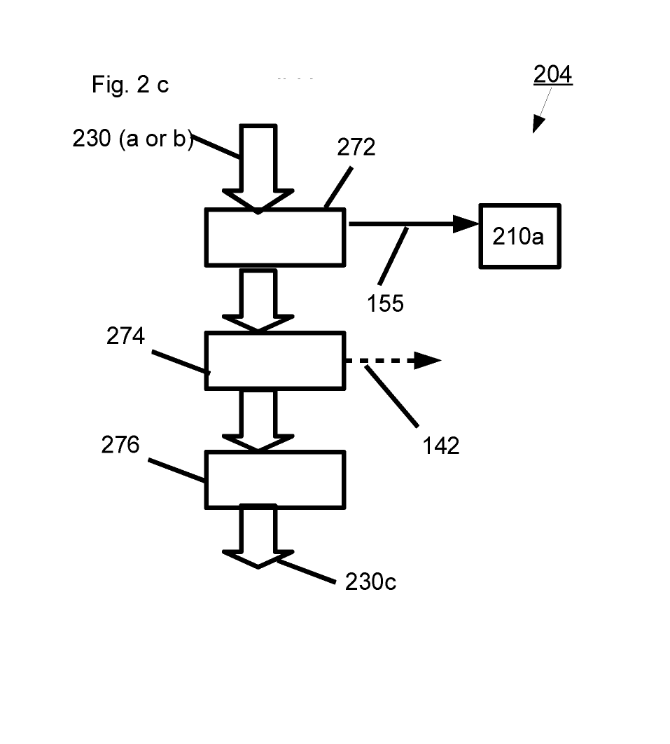

FIG. 2a is a schematic representation of an exemplary embodiment of a sugar refining module indicated generally as 201. In the context of system 100, module 201 is analogous to module 200. This specification refers to HCl as an exemplary acid, although other acids could be employed. Reference is made specifically to HCl as an example throughout the specification in order to permit presentation of quantitative data. Substitution of another acid (e.g. sulfuric acid) may change relative percentages, but would not be expected to alter the underlying operational principles.

Module 201 can be described as a system including an acid extractor 210 and a chromatography component 270. In some exemplary embodiments of the invention, chromatography component 270 employs simulated moving bed (SMB) and/or sequential simulated moving bed (SSMB) technology. In some exemplary embodiments of the invention, 12 columns operating in an SSMB mode are used. In other exemplary embodiments of the invention, larger or smaller numbers of columns are employed.

Depicted exemplary acid extractor 210 is adapted to extract acid from an input stream 130 of at least 20% sugar in an aqueous solution of HCl[HCl+water] by weight. In some embodiments, adaptation includes regulation of relative flow rates and/or extractant composition. In some exemplary embodiments of the invention, extraction is with an extractant including an S1 solvent (as defined hereinabove) to produce an output sugar stream 131. In FIG. 2a the extractant is depicted as solvent 155.

Chromatography component 270 is adapted to separate residual acid from sugars in input stream 130 and produce an acid depleted sugar stream 230a. In some exemplary embodiments of the invention, chromatography component 270 includes an ion exchange resin. Exemplary adaptations include, but are not limited to resin choice, flow rate and elution conditions.

Optionally, acid extractor 210 produces a counter current flow between input stream 130 and extractant including solvent 155. At some point during the extraction, HCl 140 (dashed arrows) is separated from stream 130 and begins to flow together with solvent 155 (solid arrows) in the extractant. In the depicted embodiment, the counter current flow is created by delivering extractant containing solvent 155 from recovery module 150 to a bottom end of acid extractor 210 while input stream 130 is delivered to a top end of acid extractor 210. In some embodiments, one or more pumps (not depicted) deliver extractant containing solvent 155 and/or input stream 130 to extractor 210. In some exemplary embodiments of the invention, acid extractor 210 includes at least one pulsed column. Optionally, the pulsed column is a Bateman pulsed column (Bateman Litwin, Netherlands).

The Bateman pulsed column includes a large diameter vertical pipe filled with alternating disc & doughnut shaped baffles which insure contact between descending stream 130 and ascending extractant 155 as they pass through the column. The solvent in extractant 155 removes at least 35, 40, 45, 50, 55, or even 60% or intermediate or greater percentages of acid 140 from stream 130.

Sugars exit extractor 210 in an acid depleted stream 131 and enter chromatography component 270 where they are subject to chromatographic separation, optionally with an ion exchange resin. The effluent 230a can be either “flow through” material or a fraction which was initially retained by the resin in 270 and subsequently eluted. In some exemplary embodiments of the invention, effluent 230a is divided into a monomer enriched cut and an oligomer enriched cut. Optionally, the oligomer enriched cut contains more acid than the monomer enriched cut.

Additional Exemplary Sugar Refining System

FIG. 2b is a schematic representation of another exemplary embodiment of a sugar refining module indicated generally as 202. In the context of system 100, module 202 is analogous to module 200.

Module 202 is similar to module 201 in that it relies upon a counter current flow of input stream 130 and an extractant containing solvent 155.

One important difference between module 202 and previously described module 201 is that acid extractor 210 includes at least two acid extractors 210a and 210b arranged in series. Optionally, each of extractors 210a and 210b includes a Bateman pulsed column as described above and the two columns together remove 85, 87, 90 or even 95% or intermediate or greater percentage's of the HCl using extractant containing solvent 155.

Because the arrangement is based upon counter current flow, extractor 210b is a “first” extractor with respect to solvent 155, and a “second” extractor with respect to sugar stream 131d.

Conversely, extractor 210a is a “first” extractor with respect to sugar stream 130, and a “second” extractor with respect to solvent 155.

The various exemplary embodiments of the invention deal with both sugar refining, and considerations relating to recycling of HCl and/or solvent. In order to prevent confusion, the following description will follow sugar stream 130 as it proceeds through module 202 to emerge as acid depleted sugar stream 230b. Ordinal numbers, where employed, will be from the standpoint of sugar stream 130.

Depicted exemplary module 202 includes an acid return loop (finely dashed arrows) which routes residual acid recovered from chromatography unit 270 back to acid extractor 210b. In the depicted embodiment, this loop is via additional components (240, 250 and 260) which are described hereinbelow.

Returning now to a sequential description of input sugar stream 130 as it moves through module 210: stream 130 flows through “first” extractor 210a and is extracted with an extractant including both an S1 solvent 155 and HCl 140. In some exemplary embodiments of the invention, stream 130 includes about 30% total sugars and about 33% HCl/[HCl+water] prior to extraction. These total sugars may include as much as 40, 50, 60 or even 70% (weight basis) oligosaccharides or intermediate or greater percentages.

In some embodiments, the sugars emerge from extractor 210a as an acid reduced stream 131a including about 33 to 35% sugars. The ratio of monomeric sugars to oligomeric sugars remains substantially unchanged at this stage. The HCl concentration has been reduced to 12 to 13% at this stage. HCl 140 and S1 solvent 155 exit extractor 210a to recovery module 150. In some exemplary embodiments of the invention, HCl 140 and S1 solvent 155 are subjected to distillation, optionally azeotropic distillation. Recovery module 150 recycles separated HCl (dashed arrow) to hydrolysis reactor 110 and sends separated solvent 155 to extractor 210b.

Acid reduced stream 131a flows to secondary, hydrolysis module 240 where it is mixed with a flow of dilute aqueous HCl (finely dashed arrow) from chromatography unit 270. This dilute aqueous HCl caries additional sugars, primarily oligomeric sugars. The effect of this mixing is that the HCl concentration is reduced to 10% or less. Optionally, the HCl concentration is greater than 6%. In some exemplary embodiments of the invention, the HCl concentration is about 7%, about 8% or about 9% or intermediate percentages after mixing at this stage. Alternatively or additionally, the total sugar concentration is reduced to below 25%, below 22% or even below 20%. According to various exemplary embodiments of the invention the sugar concentration is maintained above 15%, above 17% or even above 19%. Optionally, the sugar concentration at this stage is between 15 to 25%, between 17 to 22% or between about 19 to 20%.

Following this mixing, the resultant sugar solution in dilute HCl is subject to a secondary hydrolysis reaction in module 240. According to various exemplary embodiments of the invention this secondary hydrolysis continues for at least 1, at least 2 or at least 3 hours or intermediate or longer times. Optionally, this secondary hydrolysis lasts 1 to 3 hours, optionally about 2 hours. In some exemplary embodiments of the invention, the temperature is maintained in the range of 90 to 100° C., optionally about 95° C. In some exemplary embodiments of the invention, the secondary hydrolysis converts at least 80%, optionally at least 85% of the total sugars to monomeric sugars. In some exemplary embodiments of the invention, the secondary hydrolysis conducted in module 240 converts 80 to 90%, optionally 85 to 88%, optionally about 86% of the oligomeric sugars to monomeric sugars.

Although a single secondary hydrolysis reactor 240 is depicted between acid extractors 210a and 210b for simplicity, one or more hydrolysis reactors 240 can be provided, with each of them disposed between any pair of acid extractors, of which there may be 3, 4, 5 or even more. Additional considerations relating to the secondary hydrolysis reaction conditions are described hereinbelow.

The resultant secondary hydrolyzate 131b leaves module 240 and proceeds to filtration unit 250.

Filtration unit 250, like secondary hydrolysis reactor 240, can be positioned between any pair of the at least two acid extractors (only 210a and 210b are depicted in the drawing). Filtration unit 250, removes fine particles from secondary hydrolyzate 131b. These particles are periodically washed off the filter and sent back to extractor 210a, optionally using a mixture of acid (e.g. HCl), S1 solvent and water.

Filtered secondary hydrolyzate 131c proceeds to evaporation unit 260. Filtered secondary hydrolyzate 131c is similar to secondary hydrolyzate 131b in terms of both HCl concentration and sugar concentration. Evaporation unit 260, like secondary hydrolysis reactor 240, can be positioned between any pair of the at least two acid extractors (only 210a and 210b are depicted in the drawing).

Evaporation unit 260 removes water from filtered secondary hydrolyzate 131c. Optionally, at least a portion of the water (142) produced by evaporation unit 260 serves as an elution flow for chromatography component 270. Evaporation of water causes both HCl concentration and sugar concentration to increase. Either of these increases in concentration can contribute to polymerization (re-oligomerization) of sugars. Exemplary ways to reduce such polymerization are discussed below in “Exemplary equilibrium considerations”.

Concentrated filtered secondary hydrolyzate 131d leaves evaporation unit 260 with at least 32%, optionally at least 35% sugars. Alternatively or additionally, concentrated filtered secondary hydrolyzate 131d leaves evaporation unit 260 with at least 10%, optionally at least 12% HCl.

According to various exemplary embodiments of the invention concentrated filtered secondary hydrolyzate 131d leaves evaporation unit 260 with 32 to 40%, optionally 35 to 37%, optionally about 36% sugar in about 10 to 18%, optionally 12 to 14%, optionally about 13% HCl.

Concentrated filtered secondary hydrolyzate 131d proceeds to extractor 210b which extracts it with fresh extractant containing S1 solvent 155 from recovery module 150. Optionally, extractor 210b includes a Bateman pulsed column as described above. The resultant extract containing HCl 140 and S1 solvent 155 continues to extractor 210a.

Extracted secondary hydrolyzate 131e proceeds to chromatography component 270, which optionally includes an ion exchange resin. Extracted secondary hydrolyzate 131e includes a lower concentration of acid than hydrolyzate 131d due to extraction of HCl in extractor 210b. In some exemplary embodiments of the invention, extracted secondary hydrolyzate 131e includes about 5 to 6% HCl. Alternatively or additionally, extracted secondary hydrolyzate 131e includes at least 35%, at least 37%, at least 40%, optionally about 42 to 44% sugars. “Exemplary equilibrium considerations” as described below continue to apply throughout this process.

Optionally, an additional evaporation (not depicted) raises the sugar concentration to 50, 52, 54, 56 or 58%.

Extracted secondary hydrolyzate 131e is fed onto the chromatography resin and eluted using an aqueous solution. In the depicted exemplary embodiment, aqueous solution 142 is delivered from evaporator 260. This elution produces an acid cut (fine dashed arrows to secondary hydrolysis module 240) and a monomer cut 230b. Chromatography component 270 removes at least 80%, in some embodiments at least 85%, in some embodiments at least 90% or more of the acid in 131e so that monomer cut 230b includes less than 15%, less than 10%, less than 11%, less than 8%, less than 7%, less than 6%, less than 5%, less than 4%, less than 3%, less than 2%, less than 1%, less than 0.5%, less than 0.1%, less than 0.05% or even substantially 0% HCl. According to various exemplary embodiments of the invention monomer cut 230b contains 73 to 80%, optionally 75 to 78%, optionally about 76 to 77% of the sugars which were originally present in mixture 130. In some exemplary embodiments of the invention, these sugars are about 90 to 95%, optionally about 92% monomeric sugars and about 5 to 10%, optionally about 8% oligomeric sugars. Any sugars that remain in the acid cut can be recovered to a great extent in subsequent rounds of recycling. Alternatively or additionally, sugars that remain in the acid cut can be converted from an oligomer rich mixture to a mixture that is primarily monomeric sugars.

Although the refining process has been described as a linear progression for the sake of clarity, in practice it can be both continuous and/or cyclical in part.

Optional Additional Refining Components

FIG. 2c depicts additional optional components of module 200 depicted generally as module 204. Optional module 204 further refines the output of module 201 (230a) and/or of module 202 (230b). Depicted exemplary module 204 includes a desolventizer 272 adapted to remove any remaining residual solvent 155 from 230a and/or 230b. This solvent can be recovered by sending it to recovery module 150, or to extraction unit 210 (210a is indicated in the drawing). The sugars continue to purification media 274 adapted to remove impurities likely to adversely affect downstream fermentation. In some exemplary embodiments of the invention, purification media 274 includes granular carbon, optionally provided in a column. Optionally, the granular carbon removes impurities including, but not limited to, color bodies, color precursors, hydroxymethylfurfural (HMF), nitrogen compounds, furfural, and proteinaceous materials. Each of these materials has the potential to inhibit fermentation.

Alternatively or additionally, purification media 274 includes an ion exchange resin. In some exemplary embodiments of the invention, ion exchange resin removes any anions and/or cations. According to various exemplary embodiments of the invention these anions and/or cations include, but are not limited to, amino acids, organic acids and mineral acids. Optionally, the ion exchange resin includes a combination of strong acid cation resin and weak base anion resins.

Alternatively or additionally, purification media 274 polishes the sugars with a mixed bed system using a combination of strong cation resin and strong base anion resin.

Elution of purification media 274 is with water 142 (see also FIG. 2b) which is optionally recovered and recycled. In some exemplary embodiments of the invention, recovery is via recovery module 150.

In some embodiments, the sugars concentration at this stage is about 34 to 36%.

In some exemplary embodiments of the invention, a concentrator 276 adapted to increase a solids content of the sugar stream is employed. Concentrator 276 optionally evaporates water. In some exemplary embodiments of the invention, resultant refined sugar output 230c is a solution of 77 to 80% sugar with 70% or more, optionally 80% or more, optionally 90% or more of the sugars present as monomers.

First Exemplary Method

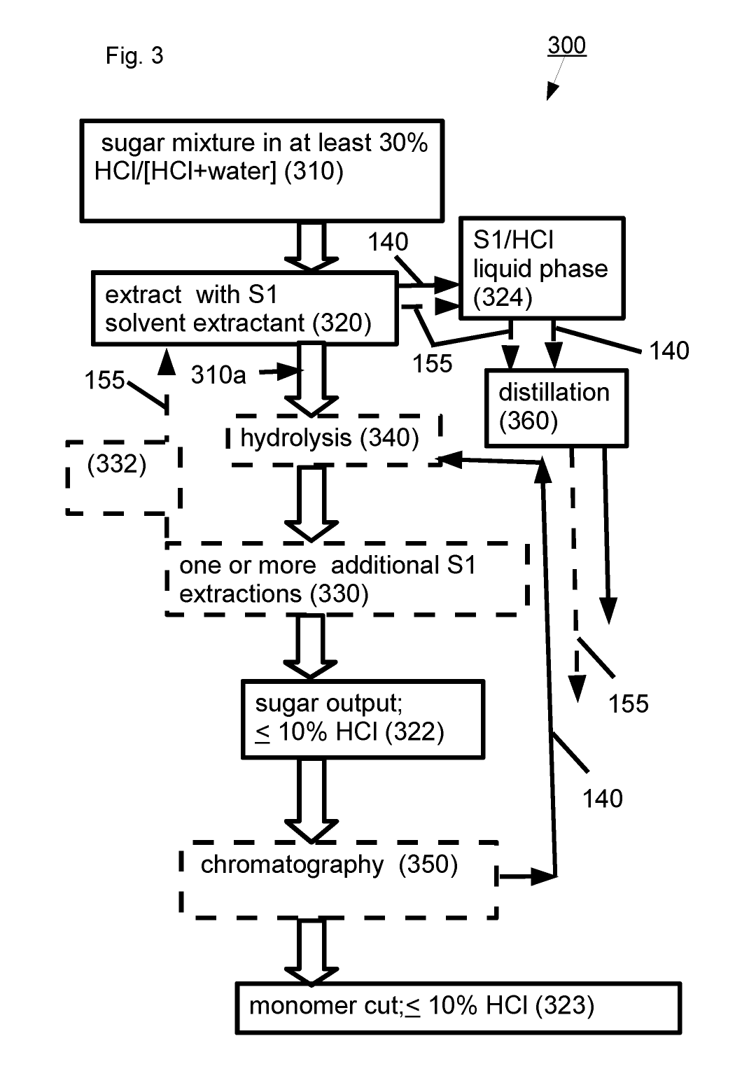

FIG. 3 is a simplified flow diagram of a method according to an exemplary embodiment of the invention depicted generally as 300. Method 300 includes extracting 320 a sugar mixture 310 in an aqueous solution of at least 30% HCl[HCl+water] with an extractant including an S1 solvent.

Depicted exemplary method 300 includes increasing a monomeric sugar to oligomeric sugar ratio in the mixture to produce a monomeric-sugar enriched mixture comprising at least 65, 70, 75 or even 80% or intermediate or greater percentages of monomeric sugars (relative to total sugars) by weight. According to various exemplary embodiments of the invention this increase may be achieved by hydrolysis 340 and/or chromatography 350 and/or additional extractions 330. In some exemplary embodiments of the invention, a combination of these techniques is employed.

Depicted exemplary method 300 includes separating an S1/HCl liquid phase 324 from mixture 310 (e.g. by extraction 320). In some embodiments, S1/HCl liquid phase 324 includes more than 20, 25, 30, 35 or even more than 40% HCl[HCl+water].

Depicted exemplary method 300 includes re-extracting 330 the monomeric-sugar enriched mixture with an extractant including an S1 solvent to produce a sugar output 322 containing less than 10% HCl/[HCl+water]. In some exemplary embodiments of the invention, creation of S1/HCl liquid phase 324 contributes to an increase in total sugar concentration in extracted sugar mixture 310a relative to original sugar mixture 310. Optionally, the total sugar concentration in 310a is 40% or more.

According to various exemplary embodiments of the invention, the S1 solvent includes n-hexanol or 2-ethyl-hexanol but not both. Optionally, one of these two solvents is combined with another S1 solvent. In some exemplary embodiments of the invention, the S1 solvent consists essentially of n-hexanol. In some exemplary embodiments of the invention, the S1 solvent consists essentially of 2-ethyl-hexanol.

Optionally, the S1 solvent includes another alcohol and/or one or more ketones and/or one or more aldehydes having at least 5 carbon atoms.

In some exemplary embodiments of the invention, the S1 solvent has a boiling point at 1 atm between 100° C. and 200° C. and forms a heterogeneous azeotrope with water, which azeotrope has a boiling point at 1 atm of less than 100° C.

In many exemplary embodiments of the invention, extracting 320 includes counter current extraction.

In some exemplary embodiments of the invention, method 300 includes one or more additional extractions 330 with an S1 containing extractant. Optionally, extraction 330 serves to reduce the HCl concentration in sugar output 322 to less than 10%.

Optionally, the monomeric-sugar enriched mixture contains ≧40% total sugars. In some exemplary embodiments of the invention, this concentration is higher than in mixture 310.

In some exemplary embodiments of the invention, hydrolysis 340 is conducted between a pair of at least two extractions. According to these embodiments, hydrolyzing 340 converts oligomeric sugars to monomeric sugars. In some embodiments, hydrolyzing 340 is conducted between a pair of at least two extraction operations (e.g. 320 and 330). Although only two extractions 320 and 330 are depicted, a larger number may actually be conducted. In some exemplary embodiments of the invention, at least one extraction conducted after hydrolysis 340 reduces an HCl concentration in the hydrolyzate. Optionally, this reduction contributes to a decrease in polymerization.

In some exemplary embodiments of the invention, extraction 320 employs an HCl containing extract 332 from a previous extraction step 330 as an extractant. As used here, the word “previous” is from the viewpoint of the S1 solvent 155 (dashed arrows), as opposed to the sugar mixture being extracted.

In some exemplary embodiments of the invention, HCl 140 is recovered (solid arrows) from chromatographic separation 350. Optionally, recovery includes routing the acid cut from a chromatography procedure (e.g. using ion exchange resin) to hydrolysis 340. Optionally, chromatography 350 employs a cation resin and/or anion resin. Exemplary resins suitable for use in various exemplary embodiments of the invention are described hereinbelow. In the depicted embodiment, chromatography 350 also produces a monomer cut 323 with an HCl concentration ≦10%. Alternatively or additionally, in some embodiments, chromatographic separation 350 produces an acid cut (indicated in the figure as 140) enriched in oligomeric sugars (in proportion to total sugars) relative to sugar mixture 322 and a monomer cut 323 enriched in monomeric sugars (in proportion to total sugars) relative to sugar mixture 322. In some embodiments, monomer cut 323 includes less than 25, less than 20, less than 15 or even 10% or less oligomeric sugars (i.e. dimers or higher) out of the total sugars. Alternatively or additionally, monomeric-sugar enriched mixture 323 includes at least 25, at least 30, at least 35 or even at least 40% total sugar by weight or intermediate or higher percentages.

In depicted exemplary method 300, HCl 140 and/or S1 155 are recovered and/or separated by distillation 360. According to various exemplary embodiments of the invention S1 155 recovered from distillation 360 is used in extraction 330 and/or 320. Alternatively or additionally, HCl 140 recovered from distillation 360 can be recycled to hydrolysis reactor 110 (FIG. 1).

Second Exemplary Method

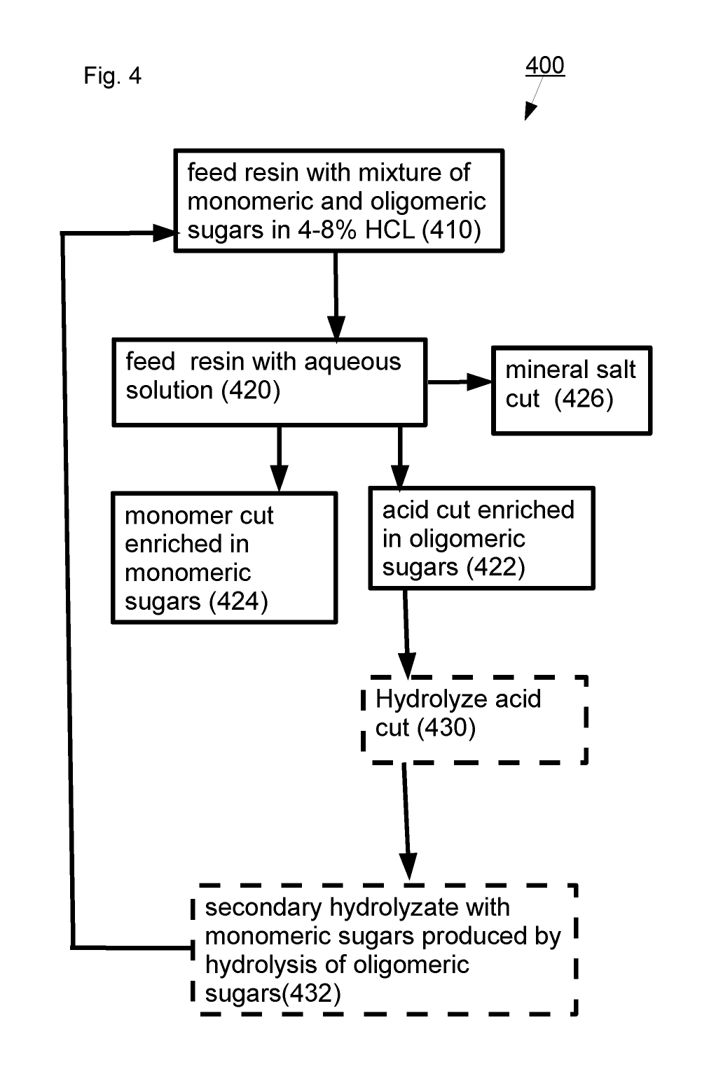

FIG. 4 is a simplified flow diagram of a method of sugar refining according to another exemplary embodiment of the invention depicted generally as 400. Method 400 includes feeding 410 a resin in a chromatographic mode with a sugar mixture including monomeric and oligomeric sugars in 4 to 8% HCl. Optionally, the HCl concentration is about 4.5 to 6.5%, optionally about 5 to 6%, optionally about 5.2 to 5.8%. In some exemplary embodiments of the invention, the sugar mixture is provided as an aqueous solution. Optionally, the mixture includes residual S1 solvent. Resins suitable for use in various exemplary embodiments of the invention are described hereinbelow in the section entitled “Exemplary Chromatography Resins”. Optionally, a strong acid cation resin is employed.

According to various exemplary embodiments of the invention the sugar mixture includes at least 40%, at least 45%, at least 50%, optionally at least 51%, at least 52%, at least 53%, or even at least 54% or intermediate or greater concentrations of total sugars. Optionally, the sugar mixture includes 52 to 63% total sugars by weight, optionally about 57 to 58%.

Depicted exemplary method 400 includes feeding 420 the resin with an aqueous solution (optionally water) to produce an acid cut 422 enriched in oligomeric sugars (in proportion to total sugars) relative to the mixture fed at 410 and a monomer cut 424 enriched in monomeric sugars (in proportion to total sugars) relative to the mixture fed at 410. Optionally, monomer cut 424 contains at least 65, 75 or even 80% monomeric sugars or intermediate or greater percentages (relative to total sugars). Optionally, acid cut 422 includes at least 10, optionally 20, optionally 30, optionally 40, optionally 50% or intermediate or greater percentages of the total sugars in sugar output 322 (FIG. 3).

Optionally, a mineral salt cut 426 is removed prior to acid cut 422.

In some exemplary embodiments of the invention, acid cut 422 is subject to adjustment. In some exemplary embodiments of the invention, adjustment includes hydrolyzing 430 oligomeric sugars in acid cut 422. Other adjustment strategies (not depicted) include, but are not limited to, concentration and/or water evaporation and/or incubation at a temperature greater than 60° C. for at least 10 minutes. In some exemplary embodiments of the invention, adjustment increases the ratio of monomers to oligomers.

In those exemplary embodiments of the invention in which adjustment include hydrolysis 430, a secondary hydrolyzate 432 enriched with monomeric sugars (relative to total sugars) is produced by hydrolysis of at least a portion of the oligomeric sugars in acid cut 422 is produced. Optionally, hydrolysis 430 is conducted together with hydrolysis 340 (FIG. 3) on a mixture of 310a (FIG. 3) and acid cut 422. Optionally, acid cut 422 dilutes sugars in 310a and improves hydrolysis kinetics. Alternatively or additionally, HCl in acid cut 422 helps drive the hydrolysis.

In some exemplary embodiments of the invention, sugars from secondary hydrolyzate 432 are used as a portion of the sugar mixture fed at 410 as indicated by the upward arrow.

In some exemplary embodiments of the invention, hydrolyzing 430 is catalyzed by HCl at a concentration of not more than 10%. Alternatively or additionally, hydrolyzing 430 is performed at a temperature not exceeding 97° C.

In some exemplary embodiments of the invention, secondary hydrolyzate 432 contains at least 80% monomeric sugars relative to the total sugar content. Alternatively or additionally, in some embodiments the total sugar content of secondary hydrolyzate 432 is at least 95, 66, 97, 98, 99, 99.5 or even 99.9% or intermediate percentages by weight of the sugar content of the mixture fed at 410.

Third Exemplary Method

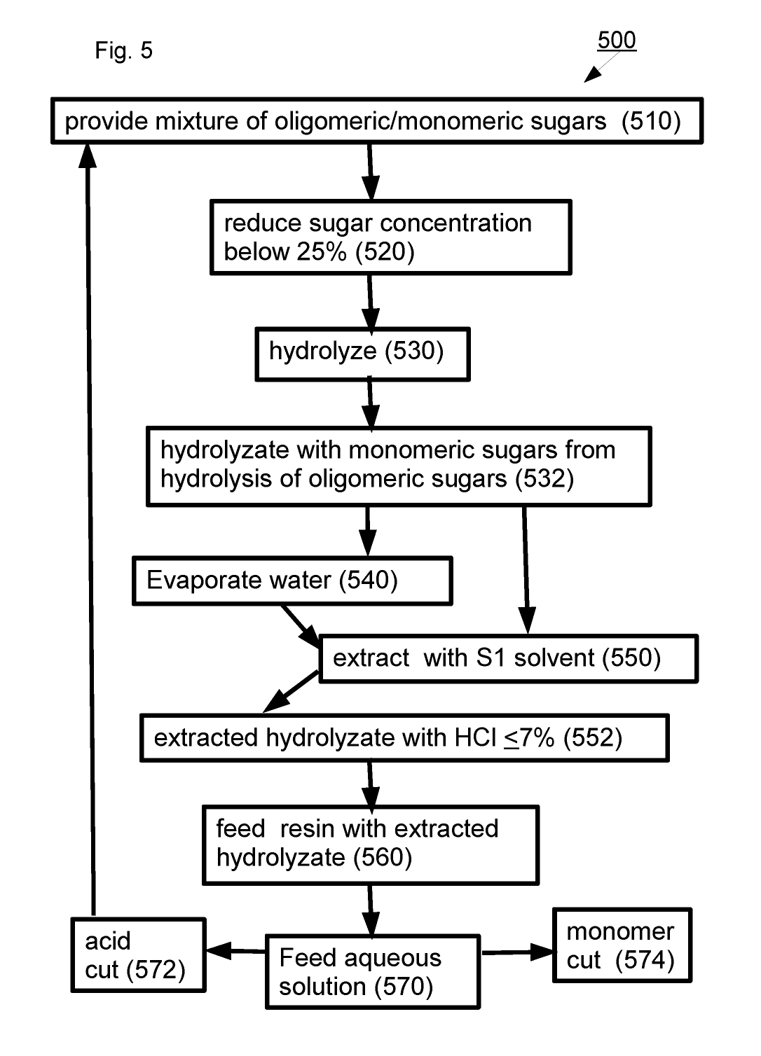

FIG. 5 is a simplified flow diagram of a sugar refining method according to another exemplary embodiment of the invention depicted generally as 500. Method 500 includes providing 510 a mixture of oligomeric and monomeric sugars at a total concentration of at least 30% in an aqueous solution of at least 10% HCl. Depicted exemplary method 500 includes reducing 520 the sugar concentration below 25%, optionally below 20%. Optionally, the reduction is sugar concentration is achieved by extraction with an extractant including an S1 solvent. Depicted exemplary method 500 also includes hydrolyzing 530 oligomeric sugars in the mixture to produce a secondary hydrolyzate 532 enriched with monomeric sugars (as a percentage of total sugars relative to mixture provided at 510). Optionally, enrichment results from hydrolysis of at least a portion of the oligomeric sugars in the mixture. Optionally, hydrolyzate 532 contains at least 80% monomeric sugars relative to the total amount of sugars therein.

In some exemplary embodiments of the invention, HCl concentration in the mixture provided at 510 can be in the range of 12 to 13%, optionally higher. In some exemplary embodiments of the invention, hydrolysis 530 is catalyzed by 7 to 10% HCl, optionally 8 to 9% HCl by weight. Optionally, reducing 520 also removes HCl. In some exemplary embodiments of the invention, hydrolyzing 530 is performed at a temperature not exceeding 97° C. Optionally, less than 1% non-hydrolytic degradation of sugars occurs during hydrolysis 530. In some embodiments, hydrolyzing 530 is catalyzed by the HCl. In some embodiments, the total sugar content of secondary hydrolyzate 532 is at least 95% by weight of the sugar content of the mixture provided at 510. In some exemplary embodiments of the invention, hydrolyzing 530 is catalyzed by HCl at a concentration of not more than 10% HCl by weight.

In some exemplary embodiments of the invention, method 500 includes evaporating 540 water from hydrolyzate 532. Optionally, at least part of this evaporation occurs at a temperature of 70° C. or less. In some exemplary embodiments of the invention, this low temperature favors an equilibrium balance with a high concentration of monomers. Optionally, at least 70% of the total sugars are monomers after evaporation 540. In some exemplary embodiments of the invention, less than 10, 5, 2.5 or even less than 1% or intermediate or lower percentages of monomeric sugars in hydrolyzate 532 oligomerize during evaporation 540.

Alternatively or additionally, in some exemplary embodiments of the invention, method 500 includes extracting 550 hydrolyzate 532 (optionally after evaporation 540) with an extractant including S1 solvent to produce an extracted hydrolyzate 552 comprising not more than 7% HCl by weight. Optionally, extraction 550 serves also to raise the sugar concentration, since some water is extracted together with the HCl. In some exemplary embodiments of the invention, extracted hydrolyzate 552 includes at least 50, optionally 52, optionally 54, optionally 56, optionally 57, optionally 58% or intermediate or greater percentages of total sugars by weight.

Depicted exemplary method 500 includes feeding 560 a resin in a chromatographic mode with extracted hydrolyzate 552 and feeding 570 said resin with an aqueous solution to produce an acid cut 572 enriched in oligomeric sugars (in proportion to total sugars) relative to extracted hydrolyzate 552 and a monomer cut 574 enriched in monomeric sugars (in proportion to total sugars) relative to extracted hydrolyzate 552. Optionally, feeding 570 an aqueous solution serves to elute the resin. In some embodiments, the resin is an ion exchange resin.

Optionally, acid cut 572 is recycled (upwards arrow) so that the mixture provided at 510 includes sugars from a previous acid cut 572.

Additional Exemplary Method

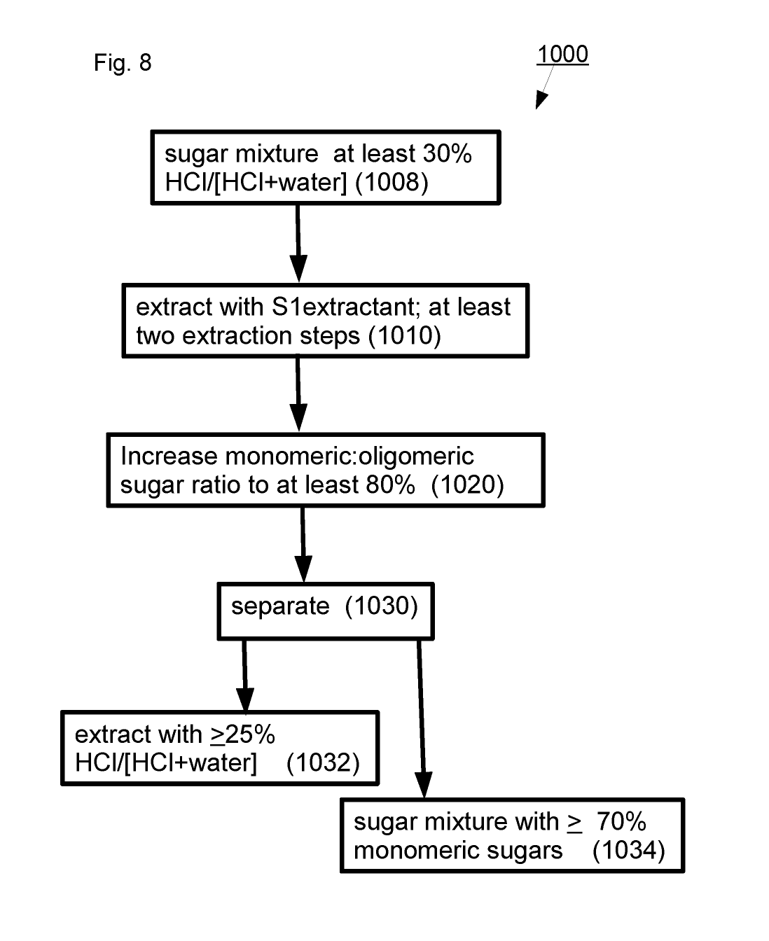

FIG. 8 is a simplified flow diagram of a method for producing a sugar mixture enriched in monomeric sugars (relative to total sugars) indicated generally as method 1000. In the depicted exemplary embodiment, method 1000 includes extracting 1010 a sugar mixture 1008 in an aqueous solution of at least 30% HCl/[HCl+water] by weight with an extractant including an S1 solvent. In some embodiments, extraction 1010 involves at least two extraction operations. Depicted exemplary method 1000 also includes increasing 1020 a monomeric sugar to oligomeric sugar ratio in mixture 1008 to produce a monomeric-sugar enriched mixture 1020 comprising at least 65, 70, 75 or 80% monomeric sugars (relative to total sugars) by weight and separating 1030 an extract 1032 having at least 25, 30, 35 or 40% or more HCl[HCl+water] and a sugar mixture 1034 having at least 60, 65, 70, 75, 80, 85 or even 90% or more monomeric sugars (relative to total sugars) and having a total sugar concentration greater than 40%. In some exemplary embodiments of the invention, increasing 1020 includes chromatographic separation as described hereinabove (e.g. 350; FIG. 3). Alternatively or additionally, in some embodiments, increasing 1020 comprises hydrolysis of oligomeric sugars as described hereinabove (e.g. 340; FIG. 3). In some embodiments increasing 1020 comprises both chromatographic separation and hydrolysis of oligomeric sugars. Alternatively or additionally, in some embodiments method 1000 includes at least one internal cycle (e.g. acid cut returning from chromatography component 270 to secondary hydrolysis reactor 240; see dotted line in FIG. 3). In some embodiments, method 1000 includes two or more internal cycles (e.g. water flowing from evaporation unit 260 to chromato-graphy component 270; see finely dashed line in FIG. 3).

Exemplary Solvent Selection Considerations

In some exemplary embodiments of the invention, extraction 320 (FIG. 3) of the sugar mixture with the S1 containing first extractant results in a selective transfer or selective extraction of HCl from the sugar mixture to the first extractant to form an S1/HCl-liquid phase (324) and an HCl-depleted sugar mixture 310a.

As used in this specification and the accompanying claims, “selective extraction of HCl” indicates extraction which is selective over water, selective over carbohydrates, or both. Again, “selective extraction of HCl” should be viewed as an example of “selective extraction of an acid”.

The selectivity of extraction of HCl over water (S_{A/W}) can be determined by equilibrating hydrolyzate with the first extractant and analyzing the concentrations of the acid and of the water in the equilibrated phases. In that case, the selectivity is:

S_{A/W}=(C_{A}/C_{W})org/(C_{A}/C_{W})aq

wherein (C_{A}/C_{W})aq is the ratio between acid concentration and water concentration in the aqueous phase and (C_{A}/C_{W})org is the ratio between acid concentration and water concentration in the organic phase.

S_{A/W} may depend on various parameters, such as temperature and the presence of other solutes in the aqueous phase, e.g. carbohydrates. Selective extraction of acid over water means S_{A/W}>1.

In some exemplary embodiments of the invention, extraction 320 of HCl from sugar mixture 310 provides, under at least some conditions, an S_{A/W} of at least about 1.1, optionally at least about 1.3 and optionally at least about 1.5.

Similarly, selectivity to acid over a carbohydrate (S_{A/C}) can be determined by equilibrating the hydrolyzate with said first extractant and analyzing the molar concentrations of the acid and the carbohydrate in the equilibrated phases. In that case, the selectivity is:

S_{A/C}=(C_{A}/C_{C})org/(C_{A}/C_{C})aq.

wherein (C_{A}/C_{C})aq is the ratio between acid concentration and the concentration of the carbohydrate (or carbohydrates) in the aqueous phase and (C_{A}/C_{C})org is the ratio of acid concentration and the concentration of the carbohydrate (or carbohydrates) in the organic phase.

S_{A/W} may depend on various parameters, such as temperature and the presence of other solutes in the aqueous phase, e.g. HCl. Selective extraction of acid over carbohydrate means S_{A/C}>1.

In some exemplary embodiments of the invention, extraction 320 of HCl from sugar mixture 310 by the first extractant has, under at least some conditions, an S_{A/C} of at least about 2, optionally at least about 5 and optionally at least about 10.

N-hexanol has a relatively high S_{A/W} and a relatively low S_{A/C}.

2-ethyl-1-hexanol has a relatively low S_{A/W} and a relatively high S_{A/C}.

These characteristics of the two hexanols caused previous efforts to use them in the context of separating sugars from HCl to focus on combining the two of them, or using one of them in combination with a complementary solvent (see for example U.S. Pat. No. 4,237,110 to Forster et al.).

According to various exemplary embodiments of the invention n-hexanol or 2-ethyl-1-hexanol is employed as the sole S1 solvent in extraction 310.

Exemplary Hydrolysis Efficiency

In some exemplary embodiments of the invention, at least 70% wt of polysaccharides in lignocellulosic substrate 112 hydrolyze into soluble carbohydrates in hydrolysis reactor 110, optionally, more than 80%, optionally more than 90%, optionally more than 95%. In some exemplary embodiments of the invention, the concentration of soluble carbohydrates in the hydrolysis medium increases with the progress of the hydrolysis reaction.

Exemplary Extractant Considerations

Optionally, the first extractant includes a mixture of an alcohol and the corresponding alkyl chloride. Optionally, the first extractant includes hexanol and hexyl chloride. Alternatively or additionally, the first extractant includes 2-ethyl-1-hexanol and 2-ethyl-1-hexyl chloride. Optionally, the first extractant includes hexanol, 2-ethyl-1-hexanol, hexyl chloride and 2-ethyl-1-hexyl chloride.

Optionally, the alcohol/alkyl chloride w/w ratio is greater than about 10 optionally greater than about 15, optionally greater than about 20, and optionally greater than about 30.

Alternatively or additionally, the first extractant also includes water.

In some exemplary embodiments of the invention, a non-carbohydrate impurity is selectively extracted into the first extractant, leading to purification of the carbohydrate in extract 310a. Optionally, the degree of selective extraction varies so that 30%, optionally 40%, optionally 50%, optionally 60%, optionally 70% or intermediate or greater percentages are achieved.

Exemplary Selective Transfer Parameters

Optionally, extraction 320 selectively transfers HCl from sugar mixture 310 to the extractant to form extract 310a and SI/HCl liquid phase 324. According to various exemplary embodiments of the invention at least 85% of the HCl transfers to the extractant, optionally at least 88%, optionally at least 92% and optionally at least 95%. In some exemplary embodiments of the invention, extract 310a contains residual HCl. Optionally, the residual HCl is equivalent to about 0.1 to about 10% of the HCl in sugar mixture 310, optionally about 0.5 to about 8% and optionally about 2 to about 7%.

Exemplary Weight Ratios

In some exemplary embodiments of the invention, the carbohydrates to water weight ratio in sugar mixture 310 is greater than 0.2, optionally greater than 0.3 and optionally greater than 0.4. Alternatively or additionally, the carbohydrates to water weight ratio in sugar mixture 310 can be less than 2.0, optionally less than 1.5 and optionally less than 1.0. Optionally, the carbohydrates to water weight ratio in sugar mixture 310 is in the range of between about 0.2 and 2.0, optionally between about 0.3 and 1.5 and optionally between 0.4 and 1.0.

In some exemplary embodiments of the invention, the HCl to water weight ratio in sugar mixture 310 is greater than 0.17, optionally greater than 0.20. Alternatively or additionally, the HCl to water weight ratio in sugar mixture 310 can be less than 0.6, optionally less 0.50. Optionally, the HCl to water weight ratio in sugar mixture 310 is in the range of between about 0.17 and 0.6, optionally between about 0.20 and 0.50.

In some exemplary embodiments of the invention, sugar mixture 310 includes about 20 to 80 weight parts of HCl and about 10 to 80 weight parts of carbohydrates per 100 weight parts of water and extract 310a includes about 3 to 35 weight parts of HCl and about 3 to 35 weight parts of water per 100 weight parts of S1.

In some exemplary embodiments of the invention, sugar mixture 310 includes about 20 to 30 weight parts of HCl and about 10 to 40 weight parts of carbohydrates per 100 weight parts of water and the HCl-carrying first extract comprises about 3 to 15 weight parts of HCl and about 2 to 20 weight parts of water per 100 weight parts of S1.

In some exemplary embodiments of the invention, sugar mixture 310 includes about 30 to 40 weight parts of HCl and about 10 to 40 weight parts of carbohydrates per 100 weight parts of water and extract 310a includes about 10 to 25 weight parts of HCl and about 10 to 25 weight parts of water per 100 weight parts of S1.

In some exemplary embodiments of the invention, sugar mixture 310 includes about 40 to 50 weight parts of HCl and about 10 to 40 weight parts of carbohydrates per 100 weight parts of water and extract 310a includes about 15 to 35 weight parts of HCl and about 15 to 35 weight parts of water per 100 weight parts of S1.

In some exemplary embodiments of the invention, sugar mixture 310 includes about 20 to 50 weight parts of HCl and about 10 to 40 weight parts of carbohydrates per 100 weight parts of water and extract 310a includes less than about 3 weight parts of carbohydrate per 100 weight parts of S1 optionally less than about 2, optionally less than about 1 and optionally less than about 0.5 weight parts of carbohydrate per 100 weight parts of S1.

In some exemplary embodiments of the invention, the total carbohydrate content in acid cut 422 is at least 10% of the carbohydrate content of the material fed at 410, optionally at least 20%, optionally at least 30% and optionally at least 40%.

In some exemplary embodiments of the invention, a total soluble carbohydrate concentration in acid cut 422 is in the range between 3% wt and 30% wt, optionally between 5% wt and 20% wt and optionally between 7% wt and 15% wt.

In some exemplary embodiments of the invention, HCl concentration in acid cut 422 is in the range between 0.5% wt and 10% wt, optionally between 1% wt and 8% wt and optionally between 3% wt and 7% wt.

Exemplary Secondary Hydrolysis Conditions

In some exemplary embodiments of the invention, hydrolysis 430 and/or 340 of oligomers in acid cut 422 is conducted at a temperature greater than 60° C., optionally between 70° C. and 130° C., optionally between 80° C. and 120° C. and optionally between 90° C. and 110° C. In some exemplary embodiments of the invention, hydrolysis 430 and/or 340 proceeds at least 10 minutes, optionally between 20 minutes and 6 hours, optionally between 30 minutes and 4 hours and optionally between 45 minutes and 2 hours.

In some exemplary embodiments of the invention, secondary hydrolysis under these conditions increases the yield of monomeric sugars with little or no degradation of sugars. In some exemplary embodiments of the invention, monomers as a fraction of total sugars is greater than 70%, optionally greater than 80%, optionally greater than 85% and optionally greater than 90% after hydrolysis 340 and/or 430. Alternatively or additionally, degradation of monomeric sugars during the hydrolysis is less than 1%, optionally less than 0.2%, optionally less than 0.1% and optionally less than 0.05%.

Exemplary Evaporation Considerations

In some exemplary embodiments of the invention, acid cut 422 is concentrated by water evaporation to reach a carbohydrate concentration of between 15% wt and 60% wt, optionally between 20% wt and 50% wt and optionally between 25% wt and 40% wt. According to various exemplary embodiments, the evaporation is conducted at reduced pressure. Alternatively or additionally, evaporation is conducted, at least partially, at a temperature lower than 100° C., optionally lower than 90° C., optionally lower than 80° C. and optionally lower than 70° C.

Exemplary Chromatography Resins

Some exemplary embodiments of the invention employ an ion exchange (1E) resin (e.g. at 410 and/or 270).

There are four main types of ion exchange resins differing in their functional groups: strongly acidic (for example using sulfonic acid groups such as sodium polystyrene sulfonate or polyAMPS), strongly basic (for example using quaternary amino groups, for example, trimethylammonium groups, e.g. polyAPTAC), weakly acidic (for example using carboxylic acid groups) and weakly basic (for example using primary, secondary and/or ternary amino groups, such as polyethylene amine).

Resins belonging to each of these four main types are commercially available. According to various exemplary embodiments of the invention, resins of one or more of these four types are employed.

In some exemplary embodiments of the invention, the resin employed at 410 (FIG. 4) and/or 270 (FIG. 2b) is a strong acid cation exchange resin in which sodium, calcium, magnesium and other cations may replace hydrogen ions on the resin due to their greater affinity for the resin than the hydrogen ion. In some exemplary embodiments, a high level of such replacement is undesirable. According to these embodiments, acid concentration in the stream fed to the chromatographic resin should be maintained sufficiently high to keep that replacement at an acceptably low level.

Strong acid cation resins include, but are not limited to, Purolite® resins such as Purolite® Resin PCR 642H+ (The Purolite Company, Bala Cynwood, Pa., USA).

In other exemplary embodiments of the invention, a dilute acid ion-exchanger is employed. According to these embodiments, acid concentration in the fed stream could be lower than when a strong acid cation exchanger is used.

In some exemplary embodiments of the invention, purification media 274 (FIG. 2c) includes a chromatographic resin. Optionally, this resin is a mixed bed system using a combination of strong cation resin and strong base anion resin. Mixed bed resins suitable for use in this context are also available from The Purolite Company (Bala Cynwood, Pa., USA).

Exemplary Equilibrium Considerations

HCl catalyzes both hydrolysis of oligomeric sugars and polymerization of monomeric sugars. Over a very long period of time, an equilibrium would be established. Reaction direction is influenced by HCl concentration, sugar concentration and ratio of monomers:oligomers. Reaction kinetics can be influenced by temperature.

Referring again to FIG. 2b and secondary hydrolysis unit 240: in some exemplary embodiments of the invention, the input sugar concentration has an excess of oligomers relative to equilibrium conditions. Dilution with the acid cut returning from chromatography unit 270 shifts the monomer:oligomer balance even further away from equilibrium conditions. Under these conditions, HCl drives the reaction in the direction of hydrolysis.

The sugar composition leaving hydrolysis unit 240 is much closer to equilibrium conditions, since oligomers have been hydrolyzed. However, subsequent filtration 250 and/or evaporation 260 shift the balance to monomeric excess. When this occurs, HCl would tend to catalyze re-polymerization of monomers to oligomers.

In order to reduce this undesired trend, in some exemplary embodiments of the invention, the sugar composition exiting hydrolysis unit 240 is cooled. According to various exemplary embodiments of the invention each ten degrees of cooling reduces the reaction kinetics by a factor of approximately 2. For example, if hydrolysis is conducted at 90° C. and the sugar composition leaving hydrolysis unit 240 is cooled to 60° C., an amount of re-polymerization would be reduced by a factor of about 8.

In some exemplary embodiments of the invention, this reduced temperature is maintained until solvent extraction 210b. Removal of (catalytic) HCl in extraction 210b also contributes to a reduction in polymerization rate.

Alternatively or additionally, a reduction in time between hydrolysis unit 240 and extraction 210b and/or chromatography 270 contributes to a reduction in polymerization.

In summary, HCl catalyzes both hydrolysis of oligomeric sugars and re-oligomerization. Hydrolysis tends to occur at lower sugars concentration and re-oligomerization becomes more likely as the sugar concentration increases. According to various exemplary embodiments of the invention hydrolysis is done at relatively low sugars concentration, but the monomeric product is concentrated after formation to facilitate acid removal. This application describes conditions under which hydrolysis, re-concentration and removal of the acid are feasible both kinetically and economically.

With regards to HCl it serves as the catalyst and its activity is concentration dependent. Hydrolysis kinetics are also improved by increasing temperature. However, HCl at too high a temperature can also catalyze degradation of sugars. The rate of such degradation increases with the proportion of monomeric sugars in the mixture. This application discloses reaction conditions which facilitate hydrolysis while limiting sugar degradation to an acceptable level.

Exemplary Flow Control Considerations

In some exemplary embodiments of the invention, liquids with varying degrees of viscosity must be transported from one module or component to another.

According to various exemplary embodiments of the invention sugar concentration and/or solvent concentration and/or HCl concentration contribute to the viscosity of a solution.

In some exemplary embodiments of the invention, this transport relies, at least partially, upon gravity. Alternatively or additionally, pumps may be employed to transport liquids.

In some exemplary embodiments of the invention, liquids move in different directions and/or at different rates. Optionally, some liquids are held in reservoirs for later use. In some exemplary embodiments of the invention, a controller serves to regulate one or more liquid flows.

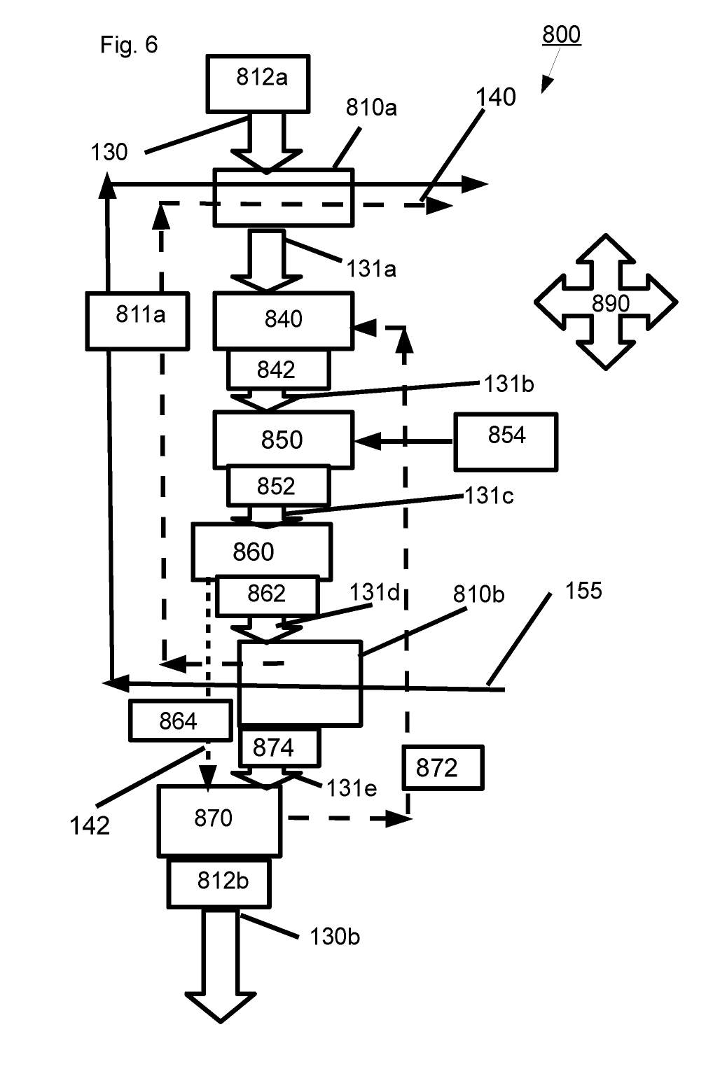

FIG. 6 is a schematic representation indicating flow control components of a sugar refining module similar to that of FIG. 2b indicated generally as 800. In the context of system 100, module 800 is analogous to module 200. Numbers beginning with the numeral “1” refer to solutions or streams described hereinabove. Many of the numbers beginning with the numeral “8” refer to similar numbers beginning with the numeral “2” in FIG. 2c and are described only in terms of their relation to flow control components here.

In the depicted embodiment, pump 811a provides a flow of S1 based extractant 155 through acid extractors 810a and 810b. The flow carries HCl 140 along with it. The extractors are arranged in series and the flow is pumped through 810b to 810a.

Pump 812a provides a flow of sugar mixture 130 to acid extractor(s) 810a. In the depicted embodiment, controller 890 regulates flow rates of pumps 812a and 811a to insure efficient extraction of acid by the extractant. Optionally, a correct relative flow rate contributes to this efficiency. In some exemplary embodiments of the invention, pumps 812a and 811a are provided as part of a Bateman pulsed column as described hereinabove. In some embodiments, flow rates in pumps 812a and/or 811a are varied to adapt acid extractor 810a to provide a desired degree of extraction efficiency.

In the depicted arrangement, acid-reduced stream 131a emerges from extractor 810a and is drawn through secondary hydrolysis module 840 by pump 842. Again, controller 890 regulates a flow rate through module 840 to insure that a desired degree of hydrolysis is achieved.

The resultant secondary hydrolyzate 131b is pumped to filtration unit 850. Optionally, filtration pump 852 draws hydrolyzate 131b through filters in the unit and/or pumps filtered secondary hydrolyzate 131c to evaporation unit 860. In the depicted arrangement, a separate pump 854 periodically provides a rinse flow (leftward pointing arrow) to filtration unit 850 to wash accumulated debris from the filters. In some exemplary embodiments of the invention, controller 890 coordinates operation of pumps 854 with 842 and/or 852 to assure proper operation of filter unit 850.

In the depicted exemplary embodiment filtered secondary hydrolyzate 131c is concentrated by evaporation unit 860, and resultant concentrated filtered secondary hydrolyzate 131d is pumped to extractor 810b by pump 862. In extractor 810b, concentrated filtered secondary hydrolyzate is extracted and the resultant extracted secondary hydrolyzate 131e is pumped to chromatography unit 870 by pump 874. As with the extraction of 810a, controller 890 coordinates operation of pumps 874 and 811a to insure a desired degree of extraction.

In the depicted exemplary arrangement, water 142 produced by evaporator 860 is pumped by collection mechanism 864 to chromatography unit 870 for use as an elution fluid. Since chromatography unit 870 cyclically alternates between sample feeding and elution in some embodiments, collection mechanism 864 optionally includes a water reservoir as well as a pump.

In the depicted exemplary arrangement, controller 890 coordinates action of collection mechanism 864 and pump 874 to cyclically feed the resin in chromatography unit 870 with a sample stream and an elution stream. This cyclic feeding and elution produces an acid cut which is recycled to hydrolysis unit 840 by pump 872 and a monomer cut 130b which is optionally pumped by pump 812b to module 204 (FIG. 2c).

Optionally, controller 890 responds to feedback from sensors (not depicted) positioned at entrances and/or exits of various units. In some exemplary embodiments of the invention, these sensors include flow sensors and controller 890 regulates relative flow rates. In some exemplary embodiments of the invention, a division between the acid cut and the monomer cut is made based upon historical performance data of the resin in chromatography unit 870 in terms of bed volumes of effluent after sample feeding.

Alternatively or additionally, the sensors include parametric detectors. Optionally, the parametric detectors monitor sugar concentration and/or acid concentration. In some exemplary embodiments of the invention, sugar concentration is measured by assaying refractive index and/or viscosity. Optionally, acid concentration is monitored by pH measurement. In some exemplary embodiments of the invention, a division between the acid cut and the monomer cut is made based upon actual performance data of the resin in chromatography unit 870 in terms of concentration of specific sugars as assayed by refractive index and/or acid concentration as estimated from pH.

Additional Exemplary Methods and Related Products

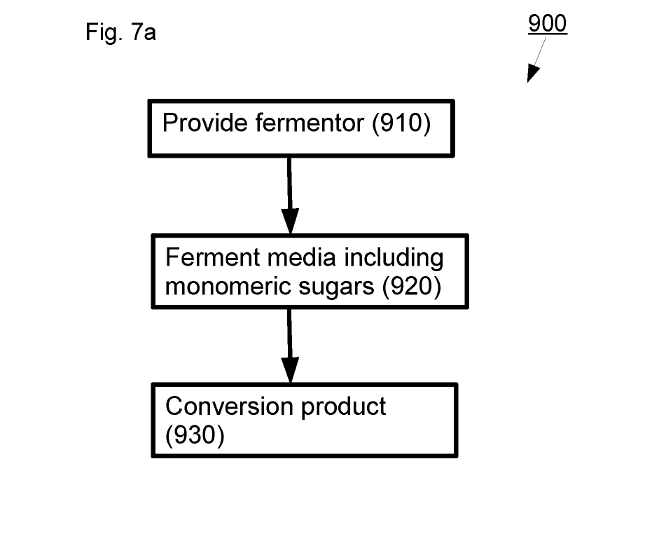

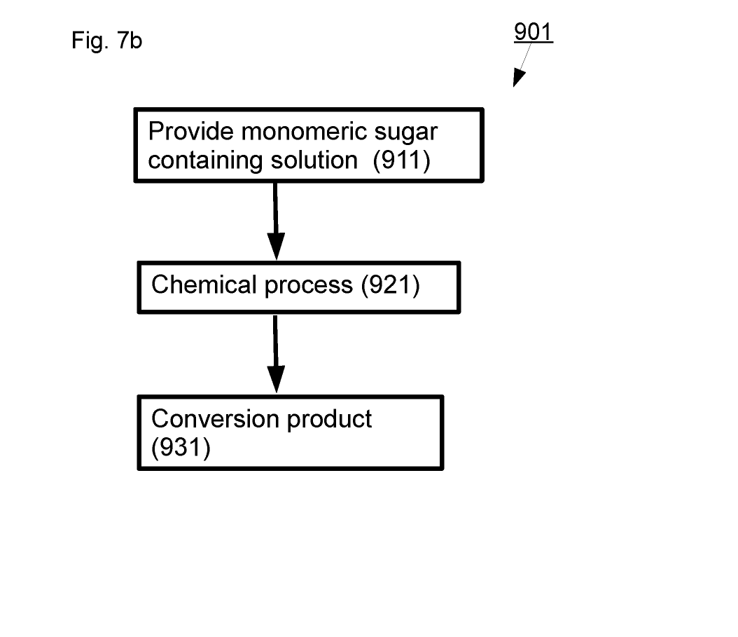

FIG. 7a is a simplified flow diagram of a method according to another exemplary embodiment of the invention depicted generally as 900. Method 900 includes providing 910 a fermentor and fermenting 920 a medium including monomeric sugars to produce a conversion product 930. In some instances processes depicted in FIGS. 1 and 2a and/or 2b and/or 2c are conducted in a single plant or system together with fermenting 920.

FIG. 7b is a simplified flow diagram of a method according to another exemplary embodiment of the invention depicted generally as 901. Method 901 includes providing 911 a monomeric sugar containing solution and converting sugars in the solution to a conversion product 931 using a chemical process 921.

According to various exemplary embodiments of the invention the monomeric sugars, or monomeric sugar containing solution, may be provided as monomeric-sugar enriched mixture (e.g. 322 or 323) and/or as a monomer cut 574 and/or as a hydrolyzate containing monomeric sugars (e.g. 510, 532 or 552).

According to various exemplary embodiments of the invention fermentation 920 and/or chemical process 921 are as described in U.S. Pat. No. 7,629,010; U.S. Pat. No. 6,833,149; U.S. Pat. No. 6,610,867; U.S. Pat. No. 6,452,051; U.S. Pat. No. 6,229,046; U.S. Pat. No. 6,207,209; U.S. Pat. No. 5,959,128; U.S. Pat. No. 5,859,270; U.S. Pat. No. 5,847,238; U.S. Pat. No. 5,602,286; and U.S. Pat. No. 5,357,035, the contents of which are incorporated by reference. In various embodiments, the processes described in the above US patents are combined with one or more methods as described herein, for example, with secondary hydrolysis and/or chromatography as described herein.

Alternatively or additionally, according to various exemplary embodiments of the invention fermentation 920 may employ a genetically modified organism (GMO). A wide range of GMOs are potentially compatible with sugars produced by the methods described herein. GMOs may include, but are not limited to, members of the genera Clostridium, Escherichia, Salmonella, Zymomonas, Rhodococcus, Pseudomonas, Bacillus, Enterococcus, Alcaligenes, Lactobacillus, Klebsiella, Paenibacillus, Corynebacterium, Brevibacterium, Pichia, Candida, Hansenula and Saccharomyces. Hosts that may be particularly of interest include Oligotropha carboxidovorans, Escherichia coli, Bacillus licheniformis, Paenibacillus macerans, Rhodococcus erythropolis, Pseudomonas putida, Lactobacillus plantarum, Enterococcus faecium, Enterococcus gallinarium, Enterococcus faecalis, Bacillus subtilis and Saccharomyces cerevisiae. Also, any of the known strains of these species may be utilized as a starting microorganism. In various exemplary embodiments, the microorganism is an actinomycete selected from Streptomyces coelicolor, Streptomyces lividans, Streptomyces hygroscopicus, or Saccharopolyspora erytraea. In various exemplary embodiments, the microorganism is a eubacterium selected from Escherichia coli, Pseudomonas fluorescens, Pseudomonas putida, Pseudomonas aeruginosa, Bacillus subtilis or Bacillus cereus.

In some exemplary embodiments, the GMO is a gram-negative bacterium. In some exemplary embodiments, the recombinant microorganism is selected from the genera Zymomonas, Escherichia, Alcaligenes and Klebsiella. In some exemplary embodiments, the recombinant microorganism is selected from the species Escherichia coli, Cupriavidus necator and Oligotropha carboxidovorans. In some exemplary embodiments, the recombinant microorganism is an E. coli strain.

In some exemplary embodiments of the invention, fermentation 920 produces lactic acid as conversion product 930. The potential of lactic acid as a commodity chemical, for example for use in the production of various industrial polymers, is known. This has been described, for example, in U.S. Pat. Nos. 5,142,023; 5,247,058; 5,258,488; 5,357,035; 5,338,822; 5,446,123; 5,539,081; 5,525,706; 5,475,080; 5,359,026; 5,484,881; 5,585,191; 5,536,807; 5,247,059; 5,274,073; 5,510,526; and 5,594,095. (The complete disclosures of these seventeen patents, which are owned by Cargill, Inc. of Minneapolis, Minn., are incorporated herein by reference.) There has been general interest in developing improved techniques for generation and isolation of lactic acid. Also, because of their potential commercial value, there is great interest in isolation of the other valuable related lactate products such as lactide, lactate esters and amides, and oligomers; see e.g. the same 17 patents.

In general, large amounts of lactic acid can be readily generated by the conduct of large-scale, industrial, microbial fermentation processes, particularly using sugars produced by exemplary methods as described herein, such as dextrose, in the media, along with suitable mineral and amino acid based nutrients. Typically, such productions occur at broth temperatures of at least 45° C., usually around 48° C.

Issues of concern with respect to lactic acid generation include, inter alia, appropriate control of pH within the fermentation system to ensure proper environment for microbial action, separation and isolation of either or both of lactic acid and lactate salts from the fermentation process and downstream isolation and production involving the isolated lactic acid or lactic acid derived product.

According to various exemplary embodiments of the invention the sugars produced by the exemplary methods described herein are incorporated into a fermentation product as described in the following US patents, the contents of each of which are hereby incorporated by reference: U.S. Pat. No. 7,678,768; U.S. Pat. No. 7,534,597; U.S. Pat. No. 7,186,856; U.S. Pat. No. 7,144,977; U.S. Pat. No. 7,019,170; U.S. Pat. No. 6,693,188; U.S. Pat. No. 6,534,679; U.S. Pat. No. 6,452,051; U.S. Pat. No. 6,361,990; U.S. Pat. No. 6,320,077; U.S. Pat. No. 6,229,046; U.S. Pat. No. 6,187,951; U.S. Pat. No. 6,160,173; U.S. Pat. No. 6,087,532; U.S. Pat. No. 5,892,109; U.S. Pat. No. 5,780,678; and U.S. Pat. No. 5,510,526.

In some exemplary embodiments of the invention, the conversion product (930 or 931) can be, for example, an alcohol, carboxylic acid, amino acid, monomer for the polymer industry or protein.

In some exemplary embodiments of the invention, the conversion product (930 or 931) is processed to produce a consumer product selected from the group consisting of a detergent, a polyethylene-based product, a polypropylene-based product, a polyolefin-based product, a polylactic acid (polylactide)-based product, a polyhydroxyalkanoate-based product and a polyacrylic-based product.

Optionally, the detergent includes a sugar-based surfactant, a fatty acid-based surfactant, a fatty alcohol-based surfactant or a cell-culture derived enzyme.

Optionally, the polyacrylic-based product is a plastic, a floor polish, a carpet, a paint, a coating, an adhesive, a dispersion, a flocculant, an elastomer, an acrylic glass, an absorbent article, an incontinence pad, a sanitary napkin, a feminine hygiene product and a diaper.

Optionally, the polyolefin-based products is a milk jug, a detergent bottle, a margarine tub, a garbage container, a plumbing pipe, an absorbent article, a diaper, a non-woven, an HDPE toy or an HDPE detergent packaging.

Optionally, the polypropylene based product is an absorbent article, a diaper or a non-woven.

Optionally, the polylactic acid based product is a packaging of an agriculture product or of a dairy product, a plastic bottle, a biodegradable product or a disposable.

Optionally, the polyhydroxyalkanoate based products is packaging of an agriculture product, a plastic bottle, a coated paper, a molded or extruded article, a feminine hygiene product, a tampon applicator, an absorbent article, a disposable non-woven or wipe, a medical surgical garment, an adhesive, an elastomer, a film, a coating, an aqueous dispersant, a fiber, an intermediate of a pharmaceutical or a binder.

Optionally, conversion product 930 or 931 is ethanol, butanol, isobutanol, a fatty acid, a fatty acid ester, a fatty alcohol or biodiesel.

In some exemplary embodiments of the invention, method 900 or 901 includes processing of conversion product 930 or 931 to produce at least one product such as, for example, an isobutene condensation product, jet fuel, gasoline, gasohol, diesel fuel, drop-in fuel, diesel fuel additive or a precursor thereof.

Optionally, the gasohol is ethanol-enriched gasoline and/or butanol-enriched gasoline.

In some exemplary embodiments of the invention, the product produced from conversion product 930 or 931 is diesel fuel, gasoline, jet fuel or a drop-in fuel.

Various exemplary embodiments of the invention include consumer products, precursors of consumer product, and ingredients of consumer products produced from conversion product 930 or 931.

Optionally, the consumer product, precursor of a consumer product, or ingredient of a consumer product includes at least one conversion product 930 or 931 such as, for example, a carboxylic or fatty acid, a dicarboxylic acid, a hydroxylcarboxylic acid, a hydroxyl di-carboxylic acid, a hydroxyl-fatty acid, methylglyoxal, mono-, di-, or poly-alcohol, an alkane, an alkene, an aromatic, an aldehyde, a ketone, an ester, a biopolymer, a protein, a peptide, an amino acid, a vitamin, an antibiotics and a pharmaceutical.

For example, the product may be ethanol-enriched gasoline, jet fuel, or biodiesel.

Optionally, the consumer product has a ratio of carbon-14 to carbon-12 of about 2.0×10⁻¹³ or greater.

Optionally, the consumer product includes an ingredient of a consumer product as described above and an additional ingredient produced from a raw material other than lignocellulosic material. In some exemplary embodiments of the invention, ingredient and the additional ingredient produced from a raw material other than lignocellulosic material are essentially of the same chemical composition.

Optionally, the consumer product includes a marker molecule at a concentration of at least 100 ppb.

According to various exemplary embodiments of the invention the marker molecule can be, for example, furfural, hydroxymethylfurfural, products of furfural or hydroxymethylfurfural condensation, color compounds derived from sugar caramelization, levulinic acid, acetic acid, methanol, galacturonic acid or glycerol.

It is expected that during the life of this patent many types of chromatography resins will be developed and the scope of the invention is intended to include all such new technologies a priori.

As used herein the term “about” refers to ±10% and includes ±1% and ±0.1%.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

Specifically, a variety of numerical indicators have been utilized. It should be understood that these numerical indicators could vary even further based upon a variety of engineering principles, materials, intended use and designs incorporated into the invention. Additionally, components and/or actions ascribed to exemplary embodiments of the invention and depicted as a single unit may be divided into subunits. Conversely, components and/or actions ascribed to exemplary embodiments of the invention and depicted as sub-units/individual actions may be combined into a single unit/action with the described/depicted function.

Alternatively, or additionally, features used to describe a method can be used to characterize an apparatus or system and features used to describe an apparatus or system can be used to characterize a method.

It should be further understood that the individual features described hereinabove can be combined in all possible combinations and sub-combinations to produce additional embodiments of the invention. The embodiments described in detail above are exemplary in nature and do not limit the scope of the invention which is defined solely by the following claims. Specifically, the invention has been described in the context of acid hydrolysis of lignocellulosic substrates but might also be used in any context in which separation of sugars from acid and/or separation of monomeric sugars from oligomeric sugars is desired.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

The terms “include”, and “have” and their conjugates as used herein mean “including but not necessarily limited to”.

Additional objects, advantages, and novel features of various embodiments of the invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non-limiting fashion.

### Example 1

### Chromatographic Separation of Acid Cut and Monomer Cut

In order to demonstrate the feasibility of separating acid from a concentrated sugar stream in dilute acid (e.g. 322 in FIG. 3), Purolite PCT 642 H+ (The Purolite Company, Bala Cynwood, Pa., USA) was used to separate an exemplary sugar feed stream in a pilot scale plant. Twelve SMB's in an SSMB configuration were used, although larger or smaller numbers of SMBs might be employed. Each column held 1 to 1.14 liters of resin. Relevant flows and their compositions are summarized in Table 1. Dissolved solids are indicated as “ds”.

TABLE 1
Exemplary chromatographic separation of sugar stream

| | Acid separation by chromatography | | | | | | | |
|---|---|---|---|---|---|---|---|---|
| | | | Elution | | | | | |
| | Feed stream | | water | | Acid cut | | | |
| Compo- | Lbs/ | | Lbs/ | | Lbs/ | | Monomer cut | |
| sition | hr | % ds | hr | % ds | hr | % ds | Lbs/hr | % ds |
| Total | 105 | | 230 | | 173.6 | | 162 | |
| Flow | | | | | | | | |
| Total | 62 | 59 | | | 18.2 | 10.5 | 43.6 | 27 |
| Solids | | | | | | | | |
| Water | 45 | | 227 | | 155 | | 117 | 72 |
| Hexanol | 0.04 | | 3.3 | | 1.9 | ~1.1 | 1.4 | <0.1 |
| Ash | .01 | ~0.1 | | | 0.1 | | <0.1 | |
| Other | .07 | | | | 0.05 | | <0.1 | |
| C6 | 55 | | | | 13.4 | | 42.6 | |
| sugars | | | | | | | | |
| C5 | 0.5 | | | | 0.1 | ~0.1 | 0.4 | |
| sugars | | | | | | | | |
| HCl | 4.6 | ~4.3 | ~0.6 | ~0.3 | 4.5 | 2.6 | ~0.6 | 0.4 |

Results presented in Table I indicate that greater than 99% of the HCl in the feed stream was separated into the acid cut. The monomer cut contains more than 75% of C6 and C5 sugars fed onto the resin in the feed stream.

This example illustrates that the acid can be separated from the majority of the sugars. As described hereinabove, the acid cut (containing the remaining sugars) can be recycled to an additional round of secondary hydrolysis.

### Example 2

### Distribution of Oligomers Between Acid Cut and Monomer Cut

In order to demonstrate the feasibility of enriching for monomeric sugars (relative to total sugars) from a concentrated sugar stream in dilute acid fraction from ion exchange resin as described in Example 1, sugars in the acid cut and monomer cut were analyzed with respect to their oligomer content. Results are summarized in Table 2.

TABLE 2
Exemplary enrichment of oligomers in acid cut

| | Oligomer enrichment | | |
|---|---|---|---|
| | Feed stream | Acid cut | Monomer cut |
| Composition | Lbs/hr | Lbs/hr | Lbs/hr |
| water | 34 | 192 | 129 |
| | (48.2%) | (89.80%) | (70.5%) |
| HCl | 5.7 | 5.0 | 0.7 |
| Total sugars | 69.9 | 16.8 | 53.1 |
| Total oligomers | 14.3 | 9.9 | 4.4 |
| (% of sugars) | (20.5) | (58.9%) | (8.3%) |

Results presented in Table 2 indicate that the monomer cut contains more than 75% of the total sugars in the feed stream, of which less than 10% are oligomeric sugars. A monomer cut stream containing more than 90% monomeric sugars (relative to total sugars) is suitable for many downstream processes including, but not limited to fermentation. The acid cut contains about 24% of the total sugars in the feed stream and is enriched in oligomeric sugars (in proportion to total sugars) relative to the feed stream.

These results confirm the acid separation results of Example 1 and demonstrate that the tested ion exchange resin also enriches for monomeric sugars (relative to total sugars) under appropriate operating conditions.

Results presented in Examples 1 and 2 also suggest that other resins, such as other strong acid cation (SAC) or weak acid cation (WAC) might be employed to give similar results.

### Example 3

### Oligomemerization Following Secondary Hydrolysis

In some embodiments, secondary hydrolysis (e.g. at 240; FIG. 2b) is implemented between a pair of extractions (e.g. 210a and 210b; FIG. 2b) to increase the proportion of monomeric sugars (relative to total sugars) in a sugar mixture. In some embodiments, evaporation (e.g. 260; FIG. 2b) is implemented to reduce a volume that must be handled by chromatographic resin (e.g. 270; FIG. 2b). However, removal of water increases a concentration of HCl. HCl can catalyze re-oligomerization of sugars as well as hydrolysis of oligomeric sugars.

Results summarized in Table 3 demonstrate that the degree of repolymerization occurring after secondary hydrolysis (e.g at 240) and prior to the second extraction is low (e.g. at 210 b).

These results demonstrate that by appropriate control of conditions, re-polymerization can be limited to acceptable levels. In some embodiments, recycling of the acid cut from the chromatographic separation to an additional round of secondary hydrolysis lessens the importance of this re-polymerization.

TABLE 3
Exemplary repolymerizatin data after secondary hydrolysis

Oligomerization

| | Post hydrolysis | Feed to second |
|---|---|---|
| | stream* | extraction** |
| Composition | Lbs/hr | Lbs/hr |
| Water | 235 | 86 |
| (% of total) | (73%) | (46%) |
| HCl | 16 | 16 |
| (% of total) | (5%) | (8.7%) |
| Total sugars | 70 | 70 |
| (% of total) | (22%) | (37.5%) |
| Total oligomers | 11 | 14.3 |
| (% of sugars) | (15.4) | (20.4%) |

*see 131b in FIG. 2b
**see 131d in FIG. 2b

1-72. (canceled)

73. A method comprising:
(a) providing a mixture of oligomeric and monomeric sugars at a total concentration of at least 30% in an aqueous solution of a dilute acid;
(b) reducing said sugar concentration below 25%; and
(c) hydrolyzing oligomeric sugars in said mixture to produce a hydrolyzate enriched with monomeric sugars (relative to total sugars).

74. A method according to claim 73, wherein said hydrolyzing is catalyzed by an acid at a concentration of not more than 10% by weight.

75. A method according to claim 73, wherein said hydrolyzing is performed at a temperature not exceeding 97° C.

76. A method according to claim 73, wherein said secondary hydrolyzate contains at least 65% by weight monomeric sugars out of total sugars.

77. A method according to claim 73, wherein the total sugar content of said secondary hydrolyzate is at least 95% by weight of the sugar content of said mixture.

78. A method according to claim 73, comprising evaporating water from said hydrolyzate at a temperature not exceeding 70° C.

79. A method according to claim 78, wherein less than 10% of monomeric sugars in said hydrolyzate oligomerize during said evaporation.

80. A method according to claim 73, comprising extracting said secondary hydrolyzate with an extractant comprising S1 solvent to produce an extracted hydrolyzate comprising not more than 7% acid by weight.

81. A method according to claim 80, wherein said extracted hydrolyzate includes at least 50%>total sugars by weight.

82. A method according to claim 80, comprising:
feeding a resin in a chromatographic mode with said extracted hydrolyzate, and feeding said resin with an aqueous solution to produce an acid cut enriched in oligomeric sugars relative to total sugars and a monomer cut enriched in monomeric sugars relative to total sugars as compared to said hydrolyzate.

83. A method according to claim 82, performed cyclically so that said mixture of oligomeric and monomeric sugars comprises sugars from a previous acid cut.
